(19) United States
(12) Patent Application Publication
(10) Pub. No.: US 2022/0218788 A1
(43) Pub. Date: Jul. 14, 2022

(54) ORALLY DELIVERED LIPID NANOPARTICLES TARGET AND REVEAL GUT CD36 AS A MASTER REGULATOR OF SYSTEMIC LIPID HOMEOSTASIS WITH DIFFERENTIAL GENDER RESPONSES

(71) Applicant: Northwestern University, Evanston, IL (US)

(72) Inventors: C. Shad Thaxton, Chicago, IL (US); Kaylin M. McMahon, Chicago, IL (US); Andrea E. Calvert, Chicago, IL (US); Timothy Feliciano, Chicago, IL (US)

(73) Assignee: Northwestern University, Evanston, IL (US)

(21) Appl. No.: 17/610,015

(22) PCT Filed: May 8, 2020
(86) PCT No.: PCT/US2020/032220

§ 371 Date: Nov. 9, 2021

Related U.S. Application Data
(60) Provisional application No. 62/846,568, filed on May 10, 2019.

(51) Int. Cl. A61K 38/17; A61K 9/00; A61K 9/127; A61K 9/16; A61K 9/50; A61K 47/28; A61P 3/04

(52) U.S. Cl. CPC A61K 38/1709; A61K 9/0053; A61K 9/127; A61P 3/04; A61K 9/5015; A61K 47/28; A61K 9/1611

(57) ABSTRACT

Disclosed herein are synthetic nanostructures, pharmaceutical compositions, kits, or methods for treating a wide spectrum of diseases associated with high fat diets or high saturated fat intake (e.g., cardiovascular disease, steatosis, cancer, diabetes type II, etc.). The synthetic nanostructures and compositions are orally administered and target and act in the gut.

### CROSS REFERENCE TO RELATED APPLICATIONS

This application claims the benefit under 35 U.S.C. § 119(e) of the filing date of U.S. Provisional Patent Application Ser. No. 62/846,568, filed May 10, 2019. The contents of the above-referenced application is hereby incorporated by reference.

### BACKGROUND

Nanoparticles are in the submicron size domain and possess unique size-dependent properties that make the materials superior compared to their bulk forms. The advanced chemical and physical properties associated with nanoparticles have led to their extensive use in the fields of biology and medicine. They have been shown to be useful for therapeutic, diagnostic, and research purposes.

Many western diets are high in saturated fat, which is among the leading killers in the world. Habitual consumption of saturated fat directly leads to diseases like cardiovascular disease, type II diabetes mellitus, fatty liver disease/non-alcoholic steatohepatitis (NASH)/cirrhosis, hypertension, etc. There is an ever-increasing need to find therapies to treat and prevent these disorders.

### SUMMARY

The present disclosure is based, at least in part, on compositions, kits, and methods for orally administering a synthetic nanostructure (e.g., HDL-NP) that targets a cell surface receptor (e.g., CD36, SR-B1) in the gut and are useful for treating a broad spectrum of diseases and bodily conditions (e.g., steatosis).

Accordingly, one aspect of the present disclosure provides a method for treating a disorder associated with high fat in a subject, comprising orally administering to the subject a synthetic nanostructure comprising a nanostructure core, an apolipoprotein, a shell comprising a lipid surrounding and attached to the nanostructure core, wherein the shell comprises a phospholipid, wherein the synthetic nanostructure is administered in an effective amount to interact with receptors in the gut endothelium, thereby treating the disorder.

In some embodiments the synthetic nanostructure comprises a nanostructure core, an apolipoprotein, a shell comprising a lipid surrounding and attached to the nanostructure core, wherein the shell comprises a phospholipid. In some embodiments, the apolipoprotein is apolipoprotein A-I, apolipoprotein A-II, or apolipoprotein E. In some embodiments, the synthetic nanostructure comprises cholesterol. In some embodiments, the shell substantially surrounds the nanostructure core. In some embodiments, the shell comprises a lipid monolayer. In some embodiments, the shell comprises a lipid bilayer. In some embodiments, the shell comprises 50-200 phospholipids. In some embodiments, the shell comprises at least 71 phospholipids. In some embodiments, the shell comprises about 71-95 phospholipids. In some embodiments, at least a portion of the lipid bilayer is covalently bound to the core. In some embodiments, the synthetic nanostructure comprises a protein associated with at least a portion of the structure. In some embodiments, the shell comprises a mixed layer of components. In some embodiments, the synthetic nanostructure has a largest cross-sectional dimension of less than or equal to about 5 nanometers (nm). In some embodiments, the nanostructure core is an inorganic nanostructure core. In some embodiments, the nanostructure core comprises gold (Au). In some embodiments, the cholesterol is esterified cholesterol. In some embodiments, the cholesterol is free cholesterol. In some embodiments, the synthetic nanostructure is administered in an effective amount to regulate systemic lipid homeostasis. In some embodiments, the disease is steatosis (fatty liver), NASH, cirrhosis; cardiovascular disease; type II DM; metabolic syndrome; depression; or steroid-based cancer. In some embodiments, the disease is non-alcoholic fatty liver disease (NAFLD). In some embodiments, the disease is not non-alcoholic fatty liver disease (NAFLD). In some embodiments, the disease is not a disease involving reverse cholesterol transport or cardiovascular disease.

In some embodiments, the disease is associated with a cancer. In some embodiments, the disease is associated with inflammation. In some embodiments, the disease is associated with prostate cancer. In some embodiments, the disease is associated with cardiovascular disease. In some embodiments, the disease is associated with nonalcoholic steatohepatitis (NASH).

In some embodiments, the methods described herein further comprise a step of identifying the subject as a subject having a disorder associated with high fat and in need of treatment with the nanostructure.

In some embodiments, the methods described herein further comprise a step of identifying the subject as a subject having a fatty liver disease and in need of treatment with the nanostructure.

In some aspects, the disclosure relates to a method for reducing fatty acid accumulation in a subject fed a high fat diet, comprising orally administering to the subject any of the synthetic HDL nanostructures of the present disclosure, wherein the synthetic HDL nanostructure is administered in an effective amount to reduce fatty acid accumulation in the subject.

In some aspects, the disclosure relates to a method for treating steatosis in a subject, comprising orally administering to the subject having steatosis, any of the synthetic HDL nanostructures of the present disclosure, wherein the synthetic HDL nanostructure is administered in an effective amount to treat steatosis in the subject.

In some aspects, the disclosure relates to a method for delivering any of the synthetic HDL nanostructures of the present disclosure locally to a gut endothelial tissue of a subject, comprising orally administering to the subject, a synthetic HDL nanostructure, wherein the local delivery of the synthetic HDL nanostructure is restricted to the gut endothelium tissue and wherein the nanostructure is not delivered systemically including to liver tissue in the subject.

In some aspects, the disclosure relates to a method for blocking fatty acid uptake by a scavenger receptor expressed on gut endothelial cells, comprising contacting the scavenger receptor with any of the synthetic HDL nanostructures of the present disclosure in the presence of fatty acids, wherein the synthetic HDL nanostructure binds to the scavenger receptor and blocks fatty acid uptake.

In some embodiments, the synthetic HDL nanostructure comprises a nanostructure core, an apolipoprotein, a shell comprising a lipid surrounding and attached to the nanostructure core, and wherein the shell comprises a phospholipid.

In some embodiments, the synthetic HDL nanostructure is administered to the subject at the same time as a fatty food. In some embodiments, the synthetic HDL nanostructure is administered to the subject within 12 hours before a fatty food. In some embodiments, the synthetic HDL nanostructure is administered to the subject within 12 hours after a fatty food. In some embodiments, the synthetic HDL nanostructure is mixed with a fatty food and administered to the subject with the fatty food. In some embodiments, the synthetic HDL nanostructure is administered to the subject within 2 hours before a fatty food. In some embodiments, the synthetic HDL nanostructure is administered to the subject within 2 hours after a fatty food. In some embodiments, the synthetic HDL nanostructure is administered to the subject once a day. In some embodiments, the synthetic HDL nanostructure is administered to the subject twice a day. In some embodiments, the synthetic HDL nanostructure is administered to the subject once every other day. In some embodiments, the synthetic HDL nanostructure is administered to the subject once a week. In some embodiments, the synthetic HDL nanostructure is administered to the subject twice a day. In some embodiments, the synthetic HDL nanostructure is administered to the subject once a day for one week to one month.

In some embodiments, the synthetic HDL nanostructure selectively binds to scavenger receptor expressed on gut endothelial cells. In some embodiments, the scavenger receptor is CD36. In some embodiments, the scavenger receptor is SR-B1.

In some embodiments, the synthetic HDL nanostructure further comprises a medicament for treating a gastrointestinal tract disorder.

In some embodiments, the methods of the present disclosure further comprise a step of identifying the subject as a subject having a disorder associated with high fat and in need of treatment with the nanostructure.

Another aspect of the present disclosure provides a pharmaceutical composition, comprising any one of the synthetic nanostructures disclosed herein and a pharmaceutically acceptable excipient. In some embodiments, the synthetic nanostructure comprises a nanostructure core, an apolipoprotein, a shell comprising a lipid surrounding and attached to the nanostructure core, wherein the shell comprises a phospholipid, and a pharmaceutically acceptable excipient formulated in an oral dosage form.

Another aspect of the present disclosure provides a liquid formulation comprising a synthetic HDL nanostructure and a liquid carrier suitable for use as an oral dosage form.

Another aspect of the present disclosure provides a solid formulation comprising a synthetic HDL nanostructure and a solid carrier suitable for use as an oral dosage form.

Another aspect of the present disclosure provides a method of treating or preventing a disease or bodily condition, comprising orally administering to a subject a therapeutically effective amount of a synthetic nanostructure or a pharmaceutical composition (as disclosed herein (e.g., HDL-NP)), thereby treating or preventing the disease or disorder. In some embodiments, the present disclosure provides for modulating fatty free acids (FFA), comprising administering a synthetic nanostructure or a pharmaceutical composition (as disclosed herein (e.g., HDL-NP)). In some embodiments, the disease or bodily condition is the result of, or associated with, a high-fat diet. In some embodiments, the FFA are the result of, or associated with, a high-fat diet.

Another aspect of the present disclosure provides a kit for treating or preventing a disease or disorder, comprising a container comprising a synthetic nanostructure or a pharmaceutical composition of any one of the preceding claims and instructions for use.

In some embodiments of the present disclosure the disease or bodily condition is associated with a cancer. In some embodiments, the disease or bodily condition is associated with inflammation. In some embodiments, the disease or bodily condition is associated with prostate cancer. In some embodiments, the disease or bodily condition is associated with steatosis. In some embodiments, the disease or bodily condition is associated with cardiovascular disease. In some embodiments, the disease or bodily condition is associated with nonalcoholic steatohepatitis (NASH). In some embodiments, the disease or bodily condition is associated with fatty liver disease. In some embodiments, the disease or bodily condition is associated with non-alcoholic fatty liver disease (NAFLD).

In some embodiments, the methods of the disclosure comprise administering any of the nanostructures of the disclosure and/or any of the pharmaceutical compositions of the disclosure to a subject topically. In some embodiments, the topical administration is to a tissue. In some embodiments, the topical administration is topically to an internal tissue. In some embodiments, the methods of the disclosure comprise administering any of the nanostructures of the disclosure and/or any of the pharmaceutical compositions of the disclosure to a subject by oral administration. In some embodiments, the oral administration facilitates administration topically to an internal tissue. In some embodiments, the oral administration facilitates coating of the gut (e.g., gastrointestinal tract) of the subject with any of the nanostructures of the disclosure and/or any of the pharmaceutical compositions of the disclosure. In some embodiments, any of the nanostructures of the disclosure and/or any of the pharmaceutical compositions of the disclosure are formulated for topical administration. In some embodiments, any of the nanostructures of the disclosure and/or any of the pharmaceutical compositions of the disclosure are formulated for oral administration. In some embodiments, any of the nanostructures of the disclosure and/or any of the pharmaceutical compositions of the disclosure are formulated into a liquid. In some embodiments, the liquid is consumed orally. In some embodiments, the liquid is encapsulated. In some embodiments, the liquid is placed into a gel capsule for consumption. In some embodiments, the liquid is in a shell for consumption. In some embodiments, the liquid is in a pill for consumption. In some embodiments, the nanostructures of the disclosure and/or any of the pharmaceutical compositions of the disclosure are formulated into a powder. In some embodiments, the powder is consumed by the subject. In some embodiments, the powder is formulated into a pill. In some embodiments, the powder is mixable with a liquid. In some embodiments, the powder is encapsulated.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. For purposes of clarity, not every component may be labeled in every drawing. It is to be understood that the data illustrated in the drawings in no way limit the scope of the disclosure. In the drawings:

FIGS. 1A-1C show an overview of High-Density Lipoprotein-Like Nanoparticles (HDL-NP). FIG. 1A shows a schematic depicting the synthesis of HDL-NPs. FIG. 1B shows a comparison of properties of HDL-NPs as compared to Native HDL. FIG. 1C shows a schematic depicting CD36-Mediated fatty free acid (FFA) trafficking in the gut.

FIGS. 2A-2B show plots of fluid intake and weight change in male and female mice over the treatment period. FIG. 2A includes plots showing the fluid intake of female (left panel) and male (right panel) mice over the treatment period. FIG. 2B includes plots showing the percent weight change of female (left panel) and male (right panel) mice measured over the treatment period. HDL-NP attenuated diet induced weight gain in male mice.

FIG. 3 includes a bar graph showing the mass of gold per liver tissue measure in the female (leftmost four columns (i.e., columns 1-4 as counted from left to right)) and male (rightmost four columns (i.e., columns 5-8 as counted from left to right)) mice to determine absorption of the HDL NPs after treatment.

FIG. 4 includes bar graphs showing the results of a serum lipid panel to measure HDL (left panel) and LDL (right panel) levels in the mice after treatment. In each panel (i.e., HDL (left panel) and LDL (right panel)), the leftmost four columns (i.e., columns 1-4 as counted from left to right) represent female mice and the rightmost four columns (i.e., columns 5-8 as counted from left to right) represent male mice.

FIG. 5 includes bar graphs showing the results of a serum lipid panel to measure total cholesterol (left panel) and free fatty acid (right panel) levels in the mice after treatment. In each panel (i.e., HDL (left panel) and LDL (right panel)), the leftmost four columns (i.e., columns 1-4 as counted from left to right) represent female mice and the rightmost four columns (i.e., columns 5-8 as counted from left to right) represent male mice.

FIGS. 6A-6D include H&E images of the stained liver tissue of female mice after the treatment period: normal chow+water (H₂O) (FIG. 6A); chow+50 nM HDL-NP in water (FIG. 6B); high-fat diet (HFD; ˜50% calories from fat)+water (FIG. 6C); HFD+50 nM HDL-NP in water (FIG. 6D). Mice were fed ad libitum for 35 days and were weighed every 3 days.

FIGS. 7A-7D include H&E images of the stained liver tissue of male mice after the treatment period: normal chow+water (H₂O) (FIG. 7A); chow+50 nM HDL-NP in water (FIG. 7B); high-fat diet (HFD; ˜50% calories from fat)+water (FIG. 7C); HFD+50 nM HDL-NP in water (FIG. 7D). Mice were fed ad libitum for 35 days and were weighed every 3 days.

FIGS. 8A-8B show oleic acid (C18:1) (FIG. 8A) and palmitic acid (C16:0) (FIG. 8B) which are the most abundant fatty acids in liver triglycerides of normal and non-alcoholic fatty liver disease (NAFLD) patients. Oleic and palmitic acids are used to generate the NAFLD model.

FIGS. 9A-9C show various aspects of the NAFLD model. FIG. 9A shows percent viability of cells exposed to the lipids (1 mM total lipids, 500 microM of each oleate and palmitate). Oleate was dissolved in 10% BSA, PBS. Palmitate was dissolved in methanol (MeOH). To ensure cells remained viable an MTS assay with varying amounts of lipids and thus varying amounts of MeOH was performed. Percent viability is on the y-axis, lipid concentration across the x-axis. FIG. 9B shows the effect on cell viability with the addition of HDL-NPs. FIG. 9C shows lipid accumulation as measured with Oil O Red.

FIGS. 10A-10F show NAFL model generation and HDL-NP modulation. FIGS. 10A-10B shows Fluorescence microscopy of Nile Red stained cells, incubated without (FIG. 10A) and with (FIG. 10B) lipids for 24 hrs. FIG. 10C shows the effect of HDL-NPs at varying concentrations as measured by Oil O Red. HDL-NPs and lipids were incubated for 24 hours (h). FIGS. 10D-10F show the difference between HepG2 cells incubated for 24 hours with and without HDL-NP as measured by confocal microscopy (FIG. 10D: without lipids, without HDL-NPs; FIG. 10E: with lipids, without HDL-NPs; FIG. 10F with lipids, with HDL-NPs).

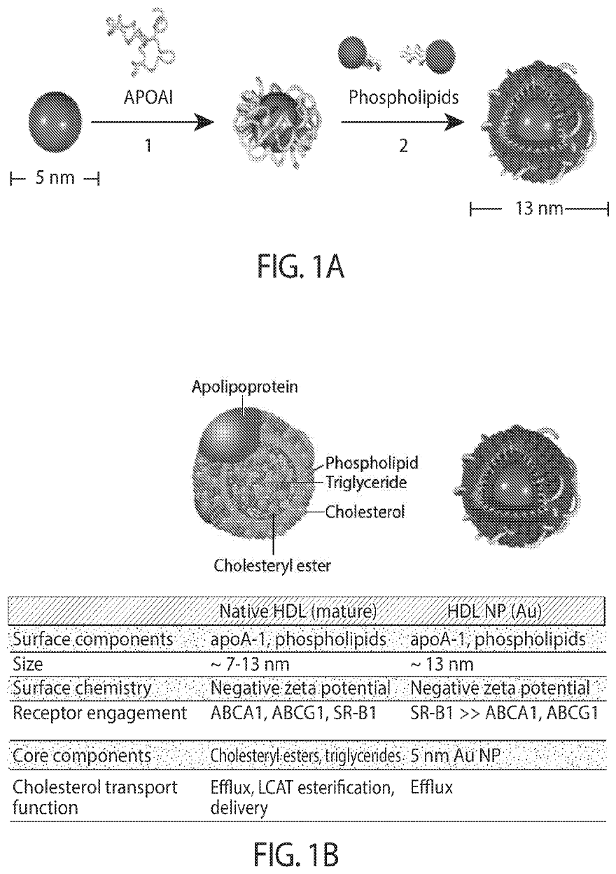

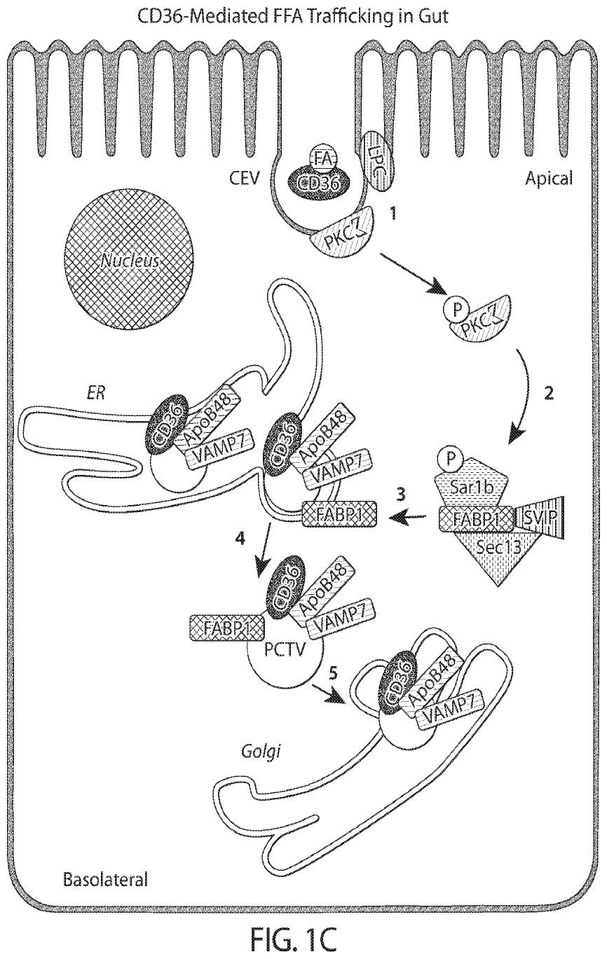

### DETAILED DESCRIPTION

The present invention relates to drugs (e.g., nanostructures, HDL-NPs) comprising high density lipoproteins nanoparticles (HDL-NPs) that are useful for the treatment of disorders associated with high fat diets or fatty free acids (FFAs). The drugs of the present invention, when administered orally, can drastically inhibit steatosis, reduce serum cholesterol and free fatty acids, and prevent weight gain and visceral fat accumulation in subjects, especially male subjects. Quite surprisingly it was discovered that synthetic HDL-NPs when administered orally are not delivered systemically to the body, but are capable of acting locally on the gut endothelium. These particles through their interaction with scavenger receptors in the gut disrupt fatty acid absorption and accumulation in the body. These findings have tremendous implications for prevention of fatty acid accumulation which leads to a number of negative health problems, without causing any systemic side effects, since the particles do not move into the systemic circulation.

The HDL-NPs and administration thereof, have a tremendous number of applications (e.g., regulating systemic lipid homeostasis). Disease processes that this drug may be useful for include, without limitation, steatosis (fatty liver), NASH, fatty liver disease, non-alcoholic fatty liver disease (NAFLD), cirrhosis; cardiovascular disease; type II DM; metabolic syndrome; depression; cancer; response to cancer therapy, like immunotherapy, etc.

The Western pattern diet (WPD) (sometime referred to as standard American diet) is a modern dietary pattern that is generally characterized by high intakes of red meat, processed meat, pre-packaged foods, butter, fried foods, high-fat dairy products, eggs, refined grains, potatoes, corn (and high-fructose corn syrup) and high-sugar drinks. It comprises a high saturated fat intake, which has become one of the major contributors to secondary diseases. The consumption of high is saturated fats is widely considered a high risk factor for several diseases, such as cardiovascular disease, dyslipidemia, type II diabetes mellitus, etc. Several studies and health reports have reported a strong correlation between saturated fat intake, blood cholesterol levels, and cardiovascular disease. Additionally, there is strong and mounting evidence that high saturated fat intake increases the risk of some cancers (e.g., prostate cancer). Further supporting this, epidemiological studies directed to the effects of the WPD have revealed elevated incidences of obesity, diabetes, cardiovascular disease-related deaths, and cancer in populations that have adopted the WPD. In 2008, the Interheart Study, funded by the Canadian Institute of Health Research, concluded that risk of a myocardial infarction is 30% higher for individuals who consume a WPD than individuals who consume a prudent diet. Despite the strong evidence of the risks associated with these high fat diets, the diets remain prevalent and are no longer limited to the Western World. Several populations are now struggling with the healthcare burden from treating the disease that stem from the high fat diets (herein referred to as secondary diseases or secondary conditions). There is an ever-increasing need to find therapies that address these secondary conditions. Further complicating the problem, there are known gender and physiologic circumstances that predispose individuals (e.g., subjects) to these secondary diseases in the presence of the high fat diet.

The present invention relates to a drug that can prevent and/or treat these secondary conditions. Such a drug would meet tremendous needs across the spectrum of health care and would have a profound societal impact. To this end, the present disclosure provides methods for the administration of a synthetic nanostructure (e.g., HDL-NP) drug to treat or prevent any on the disorders disclosed herein. Herein, it was shown that the HDL-NPs would bind to gut cell surface receptors (e.g., CD36, SR-B1). The results herein suggest that, indeed, lipid nanoparticles (e.g., HDL-NPs) target gut cell receptors (e.g., CD36, SR-B1) and reveal these receptors as critical mediators of systemic lipid homeostasis. Unexpectedly, the administration of the synthetic nanostructures of the present disclosure resulted in complete resolution of steatosis in male subjects (e.g., male mice), while female subjects (e.g., female mice) did not exhibit the same response. Furthermore, weight gain, in particular visceral fat, was found to be completely abolished in the high fat diet-fed male subjects (e.g., male mice). Peripheral blood lipids show a general decline in male subjects, especially free fatty acids and total cholesterol. It was also found that the HDL-NP drug of the present disclosure was not systemically absorbed and hepatically distributed (e.g., distributed by the liver system), which indicated that the systemic effect of the drug is due to local delivery of the drug to the gut. Interestingly, because of the gender differences, the data collected herein also suggested that that the gut cell surface receptors (e.g., CD36, SR-B1) in the gut are steroid responsive.

The compositions of the present disclosure allow for targeted delivery to the gut when administered orally. In some embodiments, the drugs are delivered orally but are not absorbed or systemically distributed to organs such as the liver. As a result, the drugs exhibit less toxicity than other drugs (e.g., non-targeted drugs) used to treat these secondary conditions. The drugs of the present disclosure can result in fewer side-effects, in part, because of the targeted delivery and action in the gut. The drugs of the present disclosure can have action that is limited to the gut (no absorption or systemic distribution) and acts upon the gut endothelium, and still effectively treat the secondary conditions. The drugs of the present disclosure are safe and highly differentiated with regard to the identified mechanisms.

The compositions of the present disclosure are targeted at a gut-expressed cell surface receptor (e.g., CD36, SR-B1). In some embodiments, the compositions of the present disclosure comprise a synthetic nanostructure that is targeted (e.g., has the ability to bind) to SR-B1, ABCA1, or ABCG1. In some embodiments, the synthetic nanostructure (e.g., HDL-NPs) is targeted to any one of the cell surface receptors in the gut. In some embodiments, the synthetic nanostructure is targeted to CD36. The synthetic nanostructures (e.g., HDL-NPs) are preferably about 5 nanometers (nm) diameter nanostructures that are surface functionalized with phospholipids and apolipoprotein A-I.

Applications

As described herein, the methods and compositions of the present invention can be used to treat or prevent several diseases (e.g., related to high fat diets, high saturated fat intake, etc.). In some embodiments, the compositions of the present disclosure are used to treat or prevent cardiovascular disease. In some embodiments, the compositions of the present disclosure are used to treat steatosis or nonalcoholic steatohepatitis (NASH). Disorders such as steatosis and NASH, there are currently no compositions approved by the United States Food and Drug Administration (FDA) for this devastating and massively prevalent condition.

Non-limiting examples of disorders that are treatable by the disclosed drugs include steatosis, steatohepatitis, nonalcoholic steatohepatitis (NASH), cirrhosis, hepatocellular carcinoma, cancer (especially hormone-driven cancers, e.g., like prostate cancer), autoimmune disease, neurological diseases (e.g., depression), type II diabetes mellitus, metabolic syndrome, diabetic nephropathy, diabetic retinopathy, macular degeneration, chronic renal insufficiency, hypertension, cardiovascular disease (e.g., MI, stroke, peripheral vascular disease), pregnancy associated fatty liver diseases (e.g., fatty liver, HELLP syndrome), pre-eclampsia, polycystic ovarian disease (PCOD), fatty liver disease, and non-alcoholic fatty liver disease (NAFLD). In some embodiments the disorder is related to fatty free acids (FFA). The compositions of the present invention can be used to treat all diseases of the innate and adaptive immune system.

Several of these diseases do not currently have approved drugs for treatment or prevention. Some of these disease have approved drugs that are systemically delivered and/or distributed, which is associated with many side effects.

The compositions of the present disclosure can be used to treat or prevent cardiovascular diseases. Non-limiting examples of examples of cardiovascular diseases and disorders are: aneurysm, stable angina, unstable angina, angina pectoris, angioneurotic edema, aortic valve stenosis, aortic aneurysm, arrhythmia, arrhythmogenic right ventricular dysplasia, arteriosclerosis, arteriovenous malformations, atrial fibrillation, Behcet syndrome, bradycardia, cardiac tamponade, cardiomegaly, congestive cardiomyopathy, hypertrophic cardiomyopathy, restrictive cardiomyopathy, carotid stenosis, cerebral hemorrhage, Churg-Strauss syndrome, diabetes, Ebstein's Anomaly, Eisenmenger complex, cholesterol embolism, bacterial endocarditis, fibromuscular dysplasia, congenital heart defects, heart diseases, congestive heart failure, heart valve diseases, heart attack, epidural hematoma, hematoma, subdural, Hippel-Lindau disease, hyperemia, hypertension, pulmonary hypertension, cardiac hypertrophy, left ventricular hypertrophy, right ventricular hypertrophy, hypoplastic left heart syndrome, hypotension, intermittent claudication, ischemic heart disease, Klippel-Trenaunay-Weber syndrome, lateral medullary syndrome, long QT syndrome mitral valve prolapse, moyamoya disease, mucocutaneous lymph node syndrome, myocardial infarction, myocardial ischemia, myocarditis, pericarditis, peripheral vascular diseases, phlebitis, polyarteritis nodosa, pulmonary atresia, Raynaud disease, Sneddon syndrome, superior vena cava syndrome, syndrome X, tachycardia, Takayasu's arteritis, hereditary hemorrhagic telangiectasia, telangiectasis, temporal arteritis, tetralogy of Fallot, thromboangiitis obliterans, thrombosis, thromboembolism, tricuspid atresia, varicose veins, vascular diseases, vasculitis, vasospasm, ventricular fibrillation, Williams syndrome, peripheral vascular disease, varicose veins and leg ulcers, deep vein thrombosis, and Wolff-Parkinson-White syndrome.

Immune disorders are broadly defined as dysfunction of the immune system (e.g., overactivity, underactivity, aberrant activity). These disorders involve fairly complex, often multiple interconnected biological pathways, which in normal physiology are critical to respond to insult or injury, initiate repair from insult or injury, and mount innate and acquired defense against foreign organisms.

In some embodiments, the compositions of the present disclosure can be used for the treatment of a subject having one or more of the immune disorders (e.g., autoimmune disorders) disclosed herein. Non-limiting examples of immune disorders include AIDS-associated myopathy, AIDS-associated neuropathy, Acute disseminated encephalomyelitis, Addison's Disease, Alopecia Areata, Anaphylaxis Reactions, Ankylosing Spondylitis, Antibody-related Neuropathies, Antiphospholipid Syndrome, Autism, Autoimmune Atherosclerosis, Autoimmune Diabetes Insipidus, Autoimmune Endometriosis, Autoimmune Eye Diseases, Autoimmune Gastritis, Autoimmune Hemolytic Anemia, Autoimmune Hemophilia, Autoimmune Hepatitis, Autoimmune Interstitial Cystitis, Autoimmune Lymphoproliferative Syndrome, Autoimmune Myelopathy, Autoimmune Myocarditis, Autoimmune Neuropathies, Autoimmune Oophoritis, Autoimmune Orchitis, Autoimmune Thrombocytopenia, Autoimmune Thyroid Diseases, Autoimmune Urticaria, Autoimmune Uveitis, Autoimmune Vasculitis, Behcet's Disease, Bell's Palsy, Bullous Pemphigoid, CREST, Celiac Disease, Cerebellar degeneration (paraneoplastic), Chronic Fatigue Syndrome, Chronic Rhinosinusitis, Chronic inflammatory demyelinating polyneuropathy, Churg Strauss Syndrome, Connective Tissue Diseases, Crohn's Disease, Cutaneous Lupus, Dermatitis Herpetiformis, Dermatomyositis, Diabetes Mellitus, Discoid Lupus Erythematosus, Drug-induced Lupus, Endocrine Orbitopathy, Glomerulonephritis, Goodpasture Syndrome, Goodpasture's Syndrome, Graves Disease, Guillian-Barre Syndrome, Miller Fisher variant of the Guillian Barre Syndrome, axonal Guillian Barre Syndrome, demyelinating Guillian Barre Syndrome, Hashimoto Thyroiditis, Herpes Gestationis, Human T-cell lymphomavirus-associated myelopathy, Huntington's Disease, IgA Nephropathy, Immune Thrombocytopenic Purpura, Inclusion body myositis, Interstitial Cystitis, Isaacs syndrome, Lambert Eaton myasthenic syndrome, Limbic encephalitis, Lower motor neuron disease, Lyme Disease, MCTD, Microscopic Polyangiitis, Miller Fisher Syndrome, Mixed Connective Tissue Disease, Mononeuritis multiplex (vasculitis), Multiple Sclerosis, Myasthenia Gravis, Myxedema, Meniere Disease, Neonatal LE, Neuropathies with dysproteinemias, Opsoclonus-myoclonus, PBC, POEMS syndrome, Paraneoplastic Autoimmune Syndromes, Pemphigus, Pemphigus foliaceus, Pemphigus vulgaris, Pernicious Anemia, Peyronie's Disease, Plasmacytoma/myeloma neuropathy, Poly-Dermatomyositis, Polyarteritis Nodosa, Polyendocrine Deficiency Syndrome, Polyendocrine Deficiency Syndrome Type 1, Polyendocrine Deficiency Syndrome Type 2, Polyglandular Autoimmune Syndrome Type I, Polyglandular Autoimmune Syndrome Type II, Polyglandular Autoimmune Syndrome Type III, Polymyositis, Primary Biliary Cirrhosis, Primary Glomerulonephritis, Primary Sclerosing Cholangitis, Psoriasis, Psoriatic Arthritis, Rasmussen's Encephalitis, Raynaud's Disease, Relapsing Polychondritis, Retrobulbar neuritis, Rheumatic Diseases, Rheumatoid Arthritis, Scleroderma, Sensory neuropathies (paraneoplastic), Sjogren's Syndrome, Stiff-Person Syndrome, Subacute Thyroiditis, Subacute autonomic neuropathy, Sydenham Chorea, Sympathetic Ophthalmitis, Systemic Lupus Erythematosus, Transverse myelitis, Type 1 Diabetes, Ulcerative Colitis, Vasculitis, Vitiligo, Wegener's Granulomatosis, Acrocyanosis, Anaphylacetic reaction, Autoimmune inner ear disease, Bilateral sensorineural hearing loss, Cold agglutinin hemolytic anemia, Cold-induced immune hemolytic anemia, Idiopathic endolymphatic hydrops, Idiopathic progressive bilateral sensorineural hearing loss, Immune-mediated inner ear disease, and Mixed autoimmune hemolysis (see for example United States Patent Application publication number US 20070202077 A1).

“Autoimmune disease,” as described herein, is a disease or disorder arising from and directed against an individual's own tissues or a co-segregate or manifestation thereof or resulting condition therefrom. Examples of autoimmune diseases or disorders include, but are not limited to, arthritis (rheumatoid arthritis, juvenile rheumatoid arthritis, osteoarthritis, psoriatic arthritis, and ankylosing spondylitis), psoriasis, dermatitis including atopic dermatitis; chronic idiopathic urticaria, polymyositis/dermatomyositis, toxic epidermal necrolysis, systemic scleroderma and sclerosis, responses associated with inflammatory bowel disease (IBD) (Crohn's disease, ulcerative colitis), respiratory distress syndrome, including adult respiratory distress syndrome (ARDS), meningitis, IgE-mediated diseases such as anaphylaxis and allergic rhinitis, encephalitis such as Rasmussen's encephalitis, uveitis, colitis such as microscopic colitis and collagenous colitis, glomerulonephritis (GN) such as membranous GN, idiopathic membranous GN, membranous proliferative GN (MPGN), including Type I and Type II, and rapidly progressive GN, allergic conditions, eczema, asthma, conditions involving infiltration of T cells and chronic inflammatory responses, atherosclerosis, autoimmune myocarditis, leukocyte adhesion deficiency, systemic lupus erythematosus (SLE) such as cutaneous SLE, lupus (including nephritis, cerebritis, pediatric, non-renal, discoid, alopecia), juvenile onset diabetes, multiple sclerosis (MS) such as spino-optical MS, allergic encephalomyelitis, immune responses associated with acute and delayed hypersensitivity mediated by cytokines and T-lymphocytes, tuberculosis, sarcoidosis, granulomatosis including Wegener's granulomatosis, agranulocytosis, vasculitis (including Large Vessel vasculitis (including Polymyalgia Rheumatica and Giant Cell (Takayasu's Arteritis), Medium Vessel vasculitis (including Kawasaki's Disease and Polyarteritis Nodosa), CNS vasculitis, and ANCA-associated vasculitis, such as Churg-Strauss vasculitis or syndrome (CSS), aplastic anemia, Coombs positive anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome, myasthenia gravis, antigen-antibody complex mediated diseases, anti-glomerular basement membrane disease, anti-phospholipid antibody syndrome, allergic neuritis, Bechet disease, Castleman's syndrome, Goodpasture's Syndrome, Lambert-Eaton Myasthenic Syndrome, Reynaud's syndrome, Sjorgen's syndrome, Stevens-Johnson syndrome, solid organ transplant rejection (including pretreatment for high panel reactive antibody titers, IgA deposit in tissues, and rejection arising from renal transplantation, liver transplantation, intestinal transplantation, cardiac transplantation, etc.), graft versus host disease (GVHD), pemphigoid bullous, pemphigus (including vulgaris, foliaceus, and pemphigus mucus-membrane pemphigoid), autoimmune polyendocrinopathies, Reiter's disease, stiff-man syndrome, immune complex nephritis, IgM polyneuropathies or IgM mediated neuropathy, idiopathic thrombocytopenic purpura (ITP), thrombotic thrombocytopenic purpura (TTP), thrombocytopenia (as developed by myocardial infarction patients, for example), including autoimmune thrombocytopenia, autoimmune disease of the testis and ovary including autoimmune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes also referred to as insulin-dependent diabetes mellitus (IDDM), including pediatric IDDM, and Sheehan's syndrome; autoimmune hepatitis, Lymphoid interstitial pneumonitis (HIV), bronchiolitis obliterans (non-transplant) vs NSIP, Guillain-Barre Syndrome, Berger's Disease (IgA nephropathy), primary biliary cirrhosis, celiac sprue (gluten enteropathy), refractory sprue with co-segregate dermatitis herpetiformis, cryoglobulinemia, amyotrophic lateral sclerosis (ALS; Lou Gehrig's disease), coronary artery disease, autoimmune inner ear disease (AIED), autoimmune hearing loss, opsoclonus myoclonus syndrome (OMS), polychondritis such as refractory polychondritis, pulmonary alveolar proteinosis, amyloidosis, giant cell hepatitis, scleritis, monoclonal gammopathy of uncertain/unknown significance (MGUS), peripheral neuropathy, paraneoplastic syndrome, channelopathies such as epilepsy, migraine, arrhythmia, muscular disorders, deafness, blindness, periodic paralysis, and channelopathies of the CNS; autism, inflammatory myopathy, and focal segmental glomerulosclerosis (FSGS).

In some embodiments, the compositions of the present invention can be used to treat or prevent cancer. In some embodiments, the cancer is characterized by cells that express scavenger receptor class B type 1 (SR-B1). In some embodiments, the cancer is a hormone-driven cancer (e.g., prostate cancer).

Non-limiting examples of cancers include: bladder cancer, breast cancer, colon and rectal cancer, endometrial cancer, kidney or renal cell cancer, leukemia, lung cancer, melanoma, Non-Hodgkin lymphoma, pancreatic cancer, prostate cancer, ovarian cancer, stomach cancer, wasting disease, and thyroid cancer. Additional non-limiting examples of cancer include Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hanlartoma, inesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinora, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis defomians), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles, dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions.

In some embodiments, the disorder associated with high fat is not a disease associated with abnormal lipid levels. A disease associated with abnormal lipid levels, as used herein, is a disease involving reverse cholesterol transport. Diseases with abnormal lipid levels are cardiovascular disease, sepsis, pancreatitis, non-alcoholic steatohepatitis, retinopathy, psoriasis, impotence, obesity, diabetes, ichtyosis, stroke, cancer, cataracts, protein storage diseases, disseminated intravascular coagulation, thrombocytopenia, rheumatic diseases, and neurological diseases.

In some embodiments the cardiovascular disease is atherosclerosis, phlebosclerosis, acute coronary syndromes, angina including, stable angina, unstable angina, inflammation, congestive heart failure, coronary heart disease (CHD), ventricular arrythmias, myocardial infarction, ischemia, coagulation disorders, thrombocytopenia, deep vein thrombosis, claudication, psoriasis, impotence, dyslipidemia, hyperlipidemia, hyperlipoproteinemia, hypoalphalipoproteinemia, hypertriglyceridemia, endothelial dysfunction, xanthomas, end organ dysfunction, vascular disease, coronary artery disease, unstable plaques, vessel intima, diseases of hemostasis, and disseminated intravascular coagulation.

As used herein, the terms “disease” and “disorder” refer to any condition that would benefit from treatment with a composition (e.g., HDL-NPs) of the present invention. This includes chronic and acute disorders or diseases including those pathological conditions that predispose the mammal to the disorder in question.

Synthetic Nanostructures

In some embodiments of the present disclosure, a synthetic nanostructure is orally administered for the treatment of the secondary conditions disclosed herein. The synthetic nanostructure may be any synthetic nanostructure having the property of being able to be bind to a cell surface receptor in the gut (e.g., CD36, SR-B1). The synthetic nanostructure may comprise a nanostructure core, a shell, the shell comprising a lipid layer surrounding and attached to the nanostructure core, and a protein associate with the shell. Examples of synthetic nanostructures useful for the present purposes are described below. In preferred embodiments, the synthetic nanostructure may be a synthetic cholesterol binding nanostructure, i.e., a biomimic of mature, spherical HDL, e.g., in terms of the size, shape, surface chemistry and/or function of the structures. Control of such features may be accomplished at least in part by using a synthetic template for the formation of the nanostructures. For example, high-density lipoprotein synthetic nanoparticles (HDL-NP) may be formed by using a gold nanoparticle (Au-NP) (or other suitable entity or material) as a synthetic template to which other components (e.g., lipids, proteins, etc.) can be added.

Examples of synthetic nanostructures that can be used in the methods are described herein. The structure (e.g., HDL-NP) has a core and a shell surrounding the core. In embodiments in which the core is a nanostructure, the core includes a surface to which one or more components can be optionally attached. For instance, in some cases, core is a nanostructure surrounded by shell, which includes an inner surface and an outer surface. The shell may be formed, at least in part, of one or more components, such as a plurality of lipids, which may optionally associate with one another and/or with surface of the core. For example, components may be associated with the core by being covalently attached to the core, physisorbed, chemisorbed, or attached to the core through ionic interactions, hydrophobic and/or hydrophilic interactions, electrostatic interactions, van der Waals interactions, or combinations thereof. In one particular embodiment, the core includes a gold nanostructure and the shell is attached to the core through a gold-thiol bond.

Optionally, components can be crosslinked to one another. Crosslinking of components of a shell can, for example, allow the control of transport of species into the shell, or between an area exterior to the shell and an area interior of the shell. For example, relatively high amounts of crosslinking may allow certain small, but not large, molecules to pass into or through the shell, whereas relatively low or no crosslinking can allow larger molecules to pass into or through the shell. Additionally, the components forming the shell may be in the form of a monolayer or a multilayer, which can also facilitate or impede the transport or sequestering of molecules. In one exemplary embodiment, shell includes a lipid bilayer that is arranged to sequester cholesterol and/or control cholesterol efflux out of cells, as described herein.

It should be understood that a shell that surrounds a core need not completely surround the core, although such embodiments may be possible. For example, the shell may surround at least 50%, at least 60%, at least 70%, at least 80%, at least 90%, or at least 99% of the surface area of a core. In some cases, the shell substantially surrounds a core. In other cases, the shell completely surrounds a core. The components of the shell may be distributed evenly across a surface of the core in some cases, and unevenly in other cases. For example, the shell may include portions (e.g., holes) that do not include any material in some cases. If desired, the shell may be designed to allow penetration and/or transport of certain molecules and components into or out of the shell, but may prevent penetration and/or transport of other molecules and components into or out of the shell. The ability of certain molecules to penetrate and/or be transported into and/or across a shell may depend on, for example, the packing density of the components forming the shell and the chemical and physical properties of the components forming the shell. As described herein, the shell may include one layer of material, or multilayers of materials in some embodiments.

In certain embodiments that synthetic nanostructure may further include one or more agents, such as a therapeutic or diagnostic agent. The agent may be a diagnostic agent (which may also be known as an imaging agent), a therapeutic agent, or both a diagnostic agent and a therapeutic agent. In certain embodiments the diagnostic agent is a tracer lipid. Tracer lipids may comprise a chromophore, a biotin subunit, or both a chromophore and a biotin subunit. The synthetic nanostructures (e.g., HDL NPs) can also be functionalized with other types of cargo such as nucleic acids. In certain embodiments the therapeutic agent may be a nucleic acid, antiviral agent, antineurological agent, antirheumatologic agent.

The one or more agents may be associated with the core, the shell, or both; e.g., they may be associated with surface of the core, inner surface of the shell, outer surface of the shell, and/or embedded in the shell. For example, one or more agents may be associated with the core, the shell, or both through covalent bonds, physisorption, chemisorption, or attached through ionic interactions, hydrophobic and/or hydrophilic interactions, electrostatic interactions, van der Waals interactions, or combinations thereof.

In some cases, the synthetic nanostructure is a synthetic cholesterol binding nanostructure having a binding constant (Kd) for cholesterol. In some embodiments, Kd is less than or equal to about 100 μM, less than or equal to about 10 μM, less than or equal to about 1 μM, less than or equal to about 0.1 μM, less than or equal to about 10 nM, less than or equal to about 7 nM, less than or equal to about 5 nM, less than or equal to about 2 nM, less than or equal to about 1 nM, less than or equal to about 0.1 nM, less than or equal to about 10 pM, less than or equal to about 1 pM, less than or equal to about 0.1 pM, less than or equal to about 10 fM, or less than or equal to about 1 fM. Methods for determining the amount of cholesterol sequestered and binding constants are known in the art.

The core of the nanostructure whether being a nanostructure core or a hollow core, may have any suitable shape and/or size. For instance, the core may be substantially spherical, non-spherical, oval, rod-shaped, pyramidal, cube-like, disk-shaped, wire-like, or irregularly shaped. In some embodiments, the core comprises a substantially spherical shape. In some embodiments, the core comprises a substantially non-spherical shape. In some embodiments, the core comprises a substantially oval shape. In some embodiments, the core comprises a substantially rod-like shape. In some embodiments, the core comprises a substantially pyramidal shape. In some embodiments, the core comprises a substantially cube-like shape. In some embodiments, the core comprises a substantially disk-like shape. In some embodiments, the core comprises a substantially wire-like shape. In some embodiments, the core comprises a substantially irregular shape. In preferred embodiments of the present invention, the core is less than or equal to about 5 nm in diameter. The core (e.g., a nanostructure core or a hollow core) may have a largest cross-sectional dimension (or, sometimes, a smallest cross-section dimension, or diameter) of, for example, less than or equal to about 500 nm, less than or equal to about 250 nm, less than or equal to about 100 nm, less than or equal to about 75 nm, less than or equal to about 50 nm, less than or equal to about 40 nm, less than or equal to about 35 nm, less than or equal to about 30 nm, less than or equal to about 25 nm, less than or equal to about 20 nm, less than or equal to about 15 nm, less than or equal to about 10 nm, less than or equal to about 5 nm, less than or equal to about 4 nm, less than or equal to about 3 nm, less than or equal to about 2 nm or less than or equal to about 1 nm. In some cases, the core has an aspect ratio of greater than about 1:1, greater than 3:1, or greater than 5:1. As used herein, “aspect ratio” refers to the ratio of a length to a width, where length and width measured perpendicular to one another, and the length refers to the longest linearly measured dimension.

In embodiments in which core includes a nanostructure core, the nanostructure core may be formed from any suitable material. In preferred embodiments, the core is formed from gold (e.g., made of gold (Au)). In some embodiments, the core is formed of a synthetic material (e.g., a material that is not naturally occurring, or naturally present in the body). In one embodiment, a nanostructure core comprises or is formed of an inorganic material. The inorganic material may include, for example, a metal (e.g., Ag, Au, Pt, Fe, Cr, Co, Ni, Cu, Zn, and other transition metals), a semiconductor (e.g., silicon, silicon compounds and alloys, cadmium selenide, cadmium sulfide, indium arsenide, and indium phosphide), or an insulator (e.g., ceramics such as silicon oxide). The inorganic material may be present in the core in any suitable amount, e.g., at least 1 percent by weight (i.e., 1 wt %), 5 wt %, 10 wt %, 25 wt %, 50 wt %, 75 wt %, 90 wt %, or 99 wt %. In one embodiment, the core is formed of 100 wt % inorganic material. The nanostructure core may, in some cases, be in the form of a quantum dot, a carbon nanotube, a carbon nanowire, or a carbon nanorod. In some cases, the nanostructure core comprises, or is formed of, a material that is not of biological origin. In some embodiments, a nanostructure includes or may be formed of one or more organic materials such as a synthetic polymer and/or a natural polymer. Examples of synthetic polymers include non-degradable polymers such as polymethacrylate and degradable polymers such as polylactic acid, polyglycolic acid and copolymers thereof. Examples of natural polymers include hyaluronic acid, chitosan, and collagen.

Furthermore, a shell of a structure can have any suitable thickness. For example, the thickness of a shell may be at least 10 Angstroms, at least 0.1 nm, at least 1 nm, at least 2 nm, at least 5 nm, at least 7 nm, at least 10 nm, at least 15 nm, at least 20 nm, at least 30 nm, at least 50 nm, at least 100 nm, or at least 200 nm (e.g., from the inner surface to the outer surface of the shell). In some cases, the thickness of a shell is less than 200 nm, less than 100 nm, less than 50 nm, less than 30 nm, less than 20 nm, less than 15 nm, less than 10 nm, less than 7 nm, less than 5 nm, less than 3 nm, less than 2 nm, or less than 1 nm (e.g., from the inner surface to the outer surface of the shell). Such thicknesses may be determined prior to or after sequestration of molecules as described herein.

Those of ordinary skill in the art are familiar with techniques to determine sizes of structures and particles. Examples of suitable techniques include dynamic light scattering (DLS) (e.g., using a Malvern Zetasizer instrument), transmission electron microscopy, scanning electron microscopy, electroresistance counting and laser diffraction. Other suitable techniques are known to those or ordinary skill in the art. Although many methods for determining sizes of nanostructures are known, the sizes described herein (e.g., largest or smallest cross-sectional dimensions, thicknesses) refer to ones measured by dynamic light scattering.

The shell of a structure described herein may comprise any suitable material, such as a hydrophobic material, a hydrophilic material, and/or an amphiphilic material. Although the shell may include one or more inorganic materials such as those listed above for the nanostructure core, in many embodiments the shell includes an organic material such as a lipid or certain polymers. The components of the shell may be chosen, in some embodiments, to facilitate the sequestering of cholesterol or other molecules. For instance, cholesterol (or other sequestered molecules) may bind or otherwise associate with a surface of the shell, or the shell may include components that allow the cholesterol to be internalized by the structure. Cholesterol (or other sequestered molecules) may also be embedded in a shell, within a layer or between two layers forming the shell.

The components of a shell may be charged, e.g., to impart a charge on the surface of the structure, or uncharged. In some embodiments, the surface of the shell may have a zeta potential of greater than or equal to about −75 mV, greater than or equal to about −60 mV, greater than or equal to about −50 mV, greater than or equal to about −40 mV, greater than or equal to about −30 mV, greater than or equal to about −20 mV, greater than or equal to about −10 mV, greater than or equal to about 0 mV, greater than or equal to about 10 mV, greater than or equal to about 20 mV, greater than or equal to about 30 mV, greater than or equal to about 40 mV, greater than or equal to about 50 mV, greater than or equal to about 60 mV, or greater than or equal to about 75 mV. The surface of the shell may have a zeta potential of less than or equal to about 75 mV, less than or equal to about 60 mV, less than or equal to about 50 mV, less than or equal to about 40 mV, less than or equal to about 30 mV, less than or equal to about 20 mV, less than or equal to about 10 mV, less than or equal to about 0 mV, less than or equal to about −10 mV, less than or equal to about −20 mV, less than or equal to about −30 mV, less than or equal to about −40 mV, less than or equal to about −50 mV, less than or equal to about −60 mV, or less than or equal to about −75 mV. Other ranges are also possible. Combinations of the above-referenced ranges are also possible (e.g., greater than or equal to about −60 mV and less than or equal to about −20 mV). As described herein, the surface charge of the shell may be tailored by varying the surface chemistry and components of the shell.

In one set of embodiments, a structure described herein or a portion thereof, such as a shell of a structure, includes one or more natural or synthetic lipids or lipid analogs (i.e., lipophilic molecules). One or more lipids and/or lipid analogues may form a single layer or a multi-layer (e.g., a bilayer) of a structure. In some instances where multi-layers are formed, the natural or synthetic lipids or lipid analogs interdigitate (e.g., between different layers). Non-limiting examples of natural or synthetic lipids or lipid analogs include fatty acyls, glycerolipids, glycerophospholipids, sphingolipids, saccharolipids and polyketides (derived from condensation of ketoacyl subunits), and sterol lipids and prenol lipids (derived from condensation of isoprene subunits).

In one particular set of embodiments, a structure described herein includes one or more phospholipids. The one or more phospholipids may include, for example, phosphatidylcholine, phosphatidylglycerol, lecithin, β, γ-dipalmitoyl-α-lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di-myrstoyl-phosphatidylserine, di-oleyl-phosphatidylcholine, 1,2-dipalmitoyl-sn-glycero-3-phosphothioethanol, and combinations thereof. In some cases, a shell (e.g., a bilayer) of a structure includes 50-200 natural or synthetic lipids or lipid analogs (e.g., phospholipids). For example, the shell may include less than about 500, less than about 400, less than about 300, less than about 200, or less than about 100 natural or synthetic lipids or lipid analogs (e.g., phospholipids), e.g., depending on the size of the structure.

Non-phosphorus containing lipids may also be used such as stearylamine, docecylamine, acetyl palmitate, and fatty acid amides. In other embodiments, other lipids such as fats, oils, waxes, cholesterol, sterols, fat-soluble vitamins (e.g., vitamins A, D, E and K), glycerides (e.g., monoglycerides, diglycerides, triglycerides) can be used to form portions of a structure described herein.

A portion of a structure described herein such as a shell or a surface of a nanostructure may optionally include one or more alkyl groups, e.g., an alkane-, alkene-, or alkyne-containing species that optionally imparts hydrophobicity to the structure. An “alkyl” group refers to a saturated aliphatic group, including a straight-chain alkyl group, branched-chain alkyl group, cycloalkyl (alicyclic) group, alkyl substituted cycloalkyl group, and cycloalkyl substituted alkyl group. The alkyl group may have various carbon numbers, e.g., between C2 and C40, and in some embodiments may be greater than C5, C10, C15, C20, C25, C30, or C35. In some embodiments, a straight chain or branched chain alkyl may have 30 or fewer carbon atoms in its backbone, and, in some cases, 20 or fewer. In some embodiments, a straight chain or branched chain alkyl may have 12 or fewer carbon atoms in its backbone (e.g., C1-C12 for straight chain, C3-C12 for branched chain), 6 or fewer, or 4 or fewer. Likewise, cycloalkyls may have from 3-10 carbon atoms in their ring structure, or 5, 6 or 7 carbons in the ring structure. Examples of alkyl groups include, but are not limited to, methyl, ethyl, propyl, isopropyl, cyclopropyl, butyl, isobutyl, tert-butyl, cyclobutyl, hexyl, cyclochexyl, and the like.

The alkyl group may include any suitable end group, e.g., a thiol group, an amino group (e.g., an unsubstituted or substituted amine), an amide group, an imine group, a carboxyl group, or a sulfate group, which may, for example, allow attachment of a ligand to a nanostructure core directly or via a linker. For example, where inert metals are used to form a nanostructure core, the alkyl species may include a thiol group to form a metal-thiol bond. In some instances, the alkyl species includes at least a second end group. For example, the species may be bound to a hydrophilic moiety such as polyethylene glycol. In other embodiments, the second end group may be a reactive group that can covalently attach to another functional group. In some instances, the second end group can participate in a ligand/receptor interaction (e.g., biotin/streptavidin).

In some embodiments, the shell includes a polymer. For example, an amphiphilic polymer may be used. The polymer may be a diblock copolymer, a triblock copolymer, etc., e.g., where one block is a hydrophobic polymer and another block is a hydrophilic polymer. For example, the polymer may be a copolymer of an α-hydroxy acid (e.g., lactic acid) and polyethylene glycol. In some cases, a shell includes a hydrophobic polymer, such as polymers that may include certain acrylics, amides and imides, carbonates, dienes, esters, ethers, fluorocarbons, olefins, sytrenes, vinyl acetals, vinyl and vinylidene chlorides, vinyl esters, vinyl ethers and ketones, and vinylpyridine and vinylpyrrolidones polymers. In other cases, a shell includes a hydrophilic polymer, such as polymers including certain acrylics, amines, ethers, styrenes, vinyl acids, and vinyl alcohols. The polymer may be charged or uncharged. As noted herein, the particular components of the shell can be chosen so as to impart certain functionality to the structures.

Where a shell includes an amphiphilic material, the material can be arranged in any suitable manner with respect to the nanostructure core and/or with each other. For instance, the amphiphilic material may include a hydrophilic group that points towards the core and a hydrophobic group that extends away from the core, or, the amphiphilic material may include a hydrophobic group that points towards the core and a hydrophilic group that extends away from the core. Bilayers of each configuration can also be formed.

The structures described herein may also include one or more proteins, polypeptides and/or peptides (e.g., synthetic peptides, amphiphilic peptides). In one set of embodiments, the structures include proteins, polypeptides and/or peptides that can increase the rate of cholesterol transfer or the cholesterol-carrying capacity of the structures. The one or more proteins or peptides may be associated with the core (e.g., a surface of the core or embedded in the core), the shell (e.g., an inner and/or outer surface of the shell, and/or embedded in the shell), or both. Associations may include covalent or non-covalent interactions (e.g., hydrophobic and/or hydrophilic interactions, electrostatic interactions, van der Waals interactions).

An example of a suitable protein that may associate with a structure described herein is an apolipoprotein, such as apolipoprotein A (e.g., apo A-I, apo A-II, apo A-IV, and apo A-V), apolipoprotein B (e.g., apo B48 and apo B100), apolipoprotein C (e.g., apo C-I, apo C-II, apo C-III, and apo C-IV), and apolipoproteins D, E, and H. Specifically, apo A1, apo A2, and apo E promote transfer of cholesterol and cholesteryl esters to the liver for metabolism and may be useful to include in structures described herein. Additionally or alternatively, a structure described herein may include one or more peptide analogues of an apolipoprotein, such as one described above. A structure may include any suitable number of, e.g., at least 1, 2, 3, 4, 5, 6, or 10, apolipoproteins or analogues thereof. In certain embodiments, a structure includes 1-6 apolipoproteins, similar to a naturally occurring HDL particle. Of course, other proteins (e.g., non-apolipoproteins) can also be included in structures described herein.

It should be understood that the components described herein, such as the lipids, phospholipids, alkyl groups, polymers, proteins, polypeptides, peptides, enzymes, bioactive agents, nucleic acids, and species for targeting described above (which may be optional), may be associated with a structure in any suitable manner and with any suitable portion of the structure, e.g., the core, the shell, or both. For example, one or more such components may be associated with a surface of a core, an interior of a core, an inner surface of a shell, an outer surface of a shell, and/or embedded in a shell. Furthermore, such components can be used, in some embodiments, to facilitate the sequestration, exchange and/or transport of materials (e.g., proteins, peptides, polypeptides, nucleic acids, nutrients) from one or more components of a subject (e.g., cells, tissues, organs, particles, fluids (e.g., blood), and portions thereof) to a structure described herein, and/or from the structure to the one or more components of the subject. In some cases, the components have chemical and/or physical properties that allow favorable interaction (e.g., binding, adsorption, transport) with the one or more materials from the subject.

Pharmaceutical Compositions

As described herein, the synthetic nanostructures may be used in “pharmaceutical compositions” or “pharmaceutically acceptable” compositions (also referred to as drugs), which comprise a therapeutically effective amount of one or more of the structures described herein, formulated together with one or more pharmaceutically acceptable carriers, additives, and/or diluents. The pharmaceutical compositions described herein may be useful for treating cancer or other conditions. It should be understood that any suitable structures described herein can be used in such pharmaceutical compositions, including those described in connection with the figures. In some cases, the structures in a pharmaceutical composition have a nanostructure core comprising an inorganic material and a shell substantially surrounding and attached to the nanostructure core.

The pharmaceutical compositions may be specially formulated for administration in solid or liquid form, including those adapted for the following: oral administration, for example, drenches (aqueous or non-aqueous solutions or suspensions), tablets, e.g., those targeted for buccal, and sublingual, boluses, powders, granules, pastes for application to the tongue; as a sterile solution or suspension, or sustained-release formulation; spray applied to the oral cavity; for example, as cream or foam.

The phrase “pharmaceutically acceptable” is employed herein to refer to those structures, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Pharmaceutical compositions described herein include those suitable for oral administration. The formulations may conveniently be presented in unit dosage form and may be prepared by any methods well known in the art of pharmacy. The amount of active ingredient that can be combined with a carrier material to produce a single dosage form will vary depending upon the host being treated, and the particular mode of administration. The amount of active ingredient that can be combined with a carrier material to produce a single dosage form will generally be that amount of the compound that produces a therapeutic effect. Generally, this amount will range from about 1% to about 99% of active ingredient, from about 5% to about 70%, or from about 10% to about 30%.

The compositions of the present disclosure (e.g., HDL-NPs, synthetic nanostructures) suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a structure described herein as an active ingredient. The compositions of the present disclosure (e.g., HDL-NPs, synthetic nanostructures) may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered structure is moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

In some embodiments an oral composition comprising the nanostructures for oral ingestion or oral administration, are formulated in and delivered with water, physiological saline or as food or a food supplement, or as a liquid supplement in encapsulated formulations.

Liquid dosage forms for oral administration of the structures described herein include pharmaceutically acceptable emulsions, microemulsions, solutions, dispersions, suspensions, syrups and elixirs. In addition to the inventive structures, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions described herein (e.g., for rectal or vaginal administration) may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the body and release the structures.

The active compound may be mixed under sterile conditions with a pharmaceutically-acceptable carrier, and with any preservatives, buffers, or propellants, which may be required.

The pastes, creams and gels may contain, in addition to the inventive structures, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the structures described herein, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Examples of suitable aqueous and nonaqueous carriers, which may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the inventive structures may be facilitated by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

Therapeutically Effective Amount

The phrase “therapeutically effective amount” as used herein means that amount of a material or composition comprising an inventive structure that is effective for producing some desired therapeutic effect in a subject at a reasonable benefit/risk ratio applicable to any medical treatment. Accordingly, a therapeutically effective amount may, for example, prevent, minimize, or reverse disease progression associated with a disease or bodily condition. Disease progression can be monitored by clinical observations, laboratory and imaging investigations apparent to a person skilled in the art. A therapeutically effective amount can be an amount that is effective in a single dose or an amount that is effective as part of a multi-dose therapy, for example an amount that is administered in two or more doses or an amount that is administered chronically.

An effective amount may depend on the particular condition to be treated. The effective amounts will depend, of course, on factors such as the severity of the condition being treated; individual patient parameters including age, physical condition, size and weight; concurrent treatments; the frequency of treatment; or the mode of administration. These factors are well known to those of ordinary skill in the art and can be addressed with no more than routine experimentation. In some cases, a maximum dose be used, that is, the highest safe dose according to sound medical judgment.

In some instances the synthetic HDL nanostructure may be administered on demand. For instance, it may be administered to the subject when the subject is consuming fatty foods. In other instances it may be administered on a regular schedule such as once a day, twice a day, once every other day, once a week, twice a day, or once a day for one week to one month. The synthetic HDL nanostructure may be mixed with or added to a food or drink product. For instance, it may be in a powder or liquid form that can be added to the food or drink. Alternatively it may be in a separate dosage form such as a capsule which can be delivered to the subject. The terms “administered” or delivered” are intended to encompass both administration by a health care worker as well as self administration by a patient.

In some embodiments a food or drink product comprising the synthetic HDL nanostructures are provided. The synthetic HDL nanostructure may be dispersed in the food or drink and the supplemented food or drink may be stored in a container.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

A physician or veterinarian having ordinary skill in the art can readily determine and prescribe the effective amount of the pharmaceutical composition required. For example, the physician or veterinarian could start doses of the structures described herein employed in the pharmaceutical composition at levels lower than that required to achieve the desired therapeutic effect and then gradually increasing the dosage until the desired effect is achieved.

The present invention also provides any of the above-mentioned compositions useful for diagnosing, preventing, treating, or managing a disease or bodily condition packaged in kits, optionally including instructions for use of the composition. That is, the kit can include a description of use of the composition for participation in any disease or bodily condition, including those associated with abnormal lipid levels. The kits can further include a description of use of the compositions as discussed herein. The kit also can include instructions for use of a combination of two or more compositions described herein. Instructions also may be provided for administering the composition by any suitable technique, such as orally, intravenously, or via another known route of drug delivery.

The kits described herein may also contain one or more containers, which can contain components such as the structures, signaling entities, and/or biomolecules as described. The kits also may contain instructions for mixing, diluting, and/or administrating the compounds. The kits also can include other containers with one or more solvents, surfactants, preservatives, and/or diluents (e.g., normal saline (0.9% NaCl), or 5% dextrose) as well as containers for mixing, diluting or administering the components to the sample or to the patient in need of such treatment.

The compositions of the kit may be provided as any suitable form, for example, as liquid solutions or as dried powders. When the composition provided is a dry powder, the powder may be reconstituted by the addition of a suitable solvent, which may also be provided. In embodiments where liquid forms of the composition are used, the liquid form may be concentrated or ready to use. The solvent will depend on the particular inventive structure and the mode of use or administration. Suitable solvents for compositions are well known and are available in the literature.

The kit, in one set of embodiments, may comprise one or more containers such as vials, tubes, and the like, each of the containers comprising one of the separate elements to be used in the method. For example, one of the containers may comprise a positive control in the assay. Additionally, the kit may include containers for other components, for example, buffers useful in the assay.

As used herein, a “subject” or a “patient” refers to any mammal (e.g., a human), for example, a mammal that may be susceptible to a disease or bodily condition such as the secondary diseases or conditions disclosed herein. Examples of subjects or patients include a human, a non-human primate, a cow, a horse, a pig, a sheep, a goat, a dog, a cat or a rodent such as a mouse, a rat, a hamster, or a guinea pig. Generally, the invention is directed toward use with humans. A subject may be a subject diagnosed with a certain disease or bodily condition or otherwise known to have a disease or bodily condition. In some embodiments, a subject may be diagnosed as, or known to be, at risk of developing a disease or bodily condition. In some embodiments, a subject may be diagnosed with, or otherwise known to have, a disease or bodily condition associated with abnormal lipid levels, as described herein. In certain embodiments, a subject may be selected for treatment on the basis of a known disease or bodily condition in the subject. In some embodiments, a subject may be selected for treatment on the basis of a suspected disease or bodily condition in the subject. In some embodiments, the composition may be administered to prevent the development of a disease or bodily condition. However, in some embodiments, the presence of an existing disease or bodily condition may be suspected, but not yet identified, and a composition of the invention may be administered to diagnose or prevent further development of the disease or bodily condition.

In some embodiments, the methods of the disclosure comprise administering any of the nanostructures of the disclosure and/or any of the pharmaceutical compositions of the disclosure to a subject topically. In some embodiments, the topical administration is to a tissue. In some embodiments, the topical administration is topically to an internal tissue. In some embodiments, the methods of the disclosure comprise administering any of the nanostructures of the disclosure and/or any of the pharmaceutical compositions of the disclosure to a subject by oral administration. In some embodiments, the oral administration facilitates administration topically to an internal tissue. In some embodiments, the oral administration facilitates coating of the gut (e.g., gastrointestinal tract) of the subject with any of the nanostructures of the disclosure and/or any of the pharmaceutical compositions of the disclosure. In some embodiments, any of the nanostructures of the disclosure and/or any of the pharmaceutical compositions of the disclosure are formulated for topical administration. In some embodiments, any of the nanostructures of the disclosure and/or any of the pharmaceutical compositions of the disclosure are formulated for oral administration. In some embodiments, any of the nanostructures of the disclosure and/or any of the pharmaceutical compositions of the disclosure are formulated into a liquid. In some embodiments, the liquid is consumed orally. In some embodiments, the liquid is encapsulated. In some embodiments, the liquid is placed into a gel capsule for consumption. In some embodiments, the liquid is in a shell for consumption. In some embodiments, the liquid is in a pill for consumption. In some embodiments, the nanostructures of the disclosure and/or any of the pharmaceutical compositions of the disclosure are formulated into a powder. In some embodiments, the powder is consumed by the subject. In some embodiments, the powder is formulated into a pill. In some embodiments, the powder is mixable with a liquid. In some embodiments, the powder is encapsulated.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

Herein, a lipid nanoparticle drug was identified that, when administered orally, drastically inhibits steatosis, reduces serum cholesterol and free fatty acids, and prevents weight gain and visceral fat accumulation in male mice fed a high fat diet over a period of five weeks. Accordingly, the lipid nanoparticle therapy has a tremendous number of applications regulating systemic lipid homeostasis. The data show complete resolution of steatosis in male mice, while female mice appear to be generally spared despite eating the same diet high in fat. Furthermore, weight gain, in particular visceral fat, is completely abolished in the high fat diet fed male mice. Peripheral blood lipids show a general decline in male mice, especially free fatty acids and total cholesterol. Finally, mass spectrometry measurement of gold in the liver reveals that the drug is not systemically absorbed and hepatically distributed indicating that the systemic effect is due to local delivery of the drug to the gut. The data implicate gut CD36 and/or SR-B1 as target(s) of orally delivered HDL NP and clearly and demonstrate a gender bias such that either or both of these receptors in the gut is regulated by androgen.

The following data reveal that male and female mice on a western style (WPD) high fat diet (HFD) demonstrate a differential response to oral administration of high density lipoprotein-like nanoparticles (HDL NP). In male mice, oral administration of HDL NPs:
Profoundly inhibits HFD-induced weight gain by reducing visceral fat accumulation
Reduces circulating free fatty acids and total cholesterol
Completely abolishes fatty liver (i.e., steatosis)
In female mice, oral administration of HDL NPs showed:
Weight gain on HFD is similar between house water and HDL NP water groups
Circulating free fatty acids and total cholesterol levels are lower than in male mice and not reduced by HDL NP
They are largely protected from HFD-induced fatty liver (i.e., steatosis) and the HDL NPs do not appear to cause a change

### Example 1

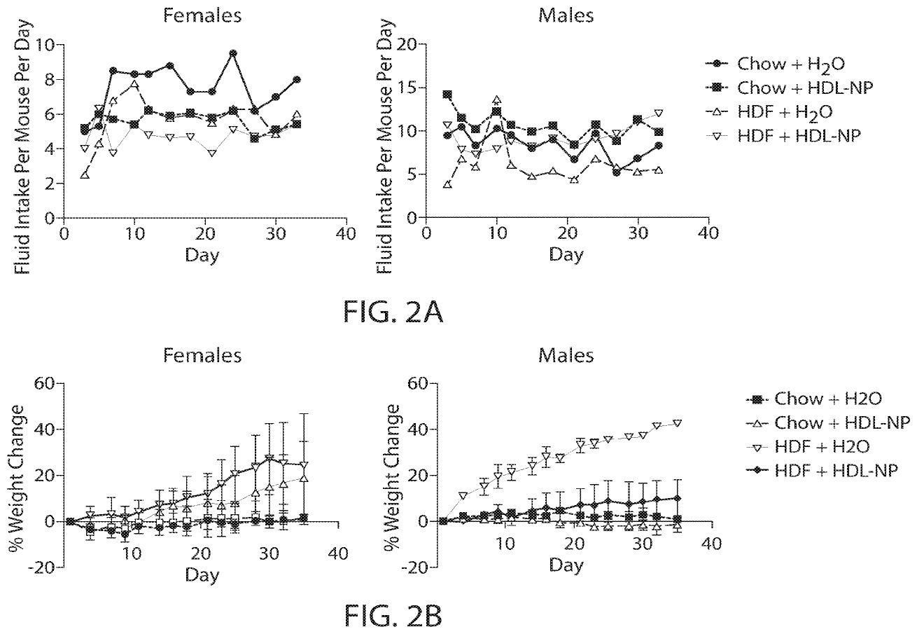

The studies were conducted by filling house water bottles with house water or house water with HDL NPs (50 nM), and then having the mice drink the provided water ad libitum for 5 weeks while being fed a high fat diet. Twenty age matched mice were arranged into 4 groups of each sex (8 groups in total), which varied their solid food (normal chow or high fat diet chow with 42% calories from fat [HFD]) and their fluid (house water alone or 50 nM HDL NP in house water). A male and a female group for each of the following were placed into separate cages: 1) normal chow+water×2 mice, 2) normal chow+LNP×3 mice, 3) HFD+water×2 mice, 4) HFD+HDL NP×3 mice. House water or HDL NP (50 nM) in house water were provided to mice ad libitum via a cage-attached bottle. Because HDL NP turn the water a light shade of red, and may impact taste fluid uptake was closely monitored throughout the experiment, and the average fluid intake per mouse per day was calculated between each measurement. It was found that HDL NP in the drinking water did not have a significant impact on fluid intake in either male or female groups. No aversion to water with HDL NP noted or measured during the study (FIG. 2A).

### Example 2

To determine the effect of oral administration of HDL NP on weight gain, mice were weighed three times per week over the course of the entire experiment (5 weeks) and the percent weight change recorded relative to their initial weight (FIG. 2B). As expected and reported, male and female mice consuming HFD and water gained considerable weight (42.1±0.71% in males and 18.9±16.1% in females, compared to 1.0±0.85% in control males and 0.95±1.9% in control females on standard chow and water alone). Strikingly, oral administration of HDL NPs to male mice nearly abolished HFD-induced weight gain in male mice (10.1±8.0%), but not in female mice (24.5±22.6%). At necropsy, visual inspection revealed that the increase in weight measured in the male mice was due to significant visceral fat accumulation that was nearly abolished by oral administration of the HDL NP. Female mice were generally spared visceral fat accumulation with and without oral HDL NP administration, and accumulated cutaneous adipose tissue.

### Example 3

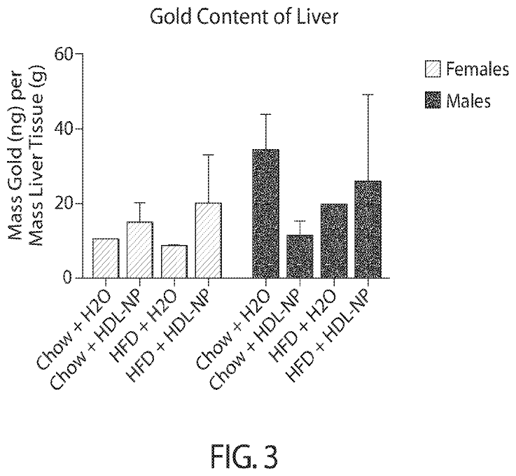

Inductively coupled plasma mass spectrometry (ICP-MS) was used to screen the liver for gold. The data revealed that the HDL NP does not gain access to the systemic circulation and reside in the liver. It is not absorbed and transported to liver after oral delivery. The background ICP-MS values are the same as those obtained when gold was orally administered (FIG. 3).

### Example 4

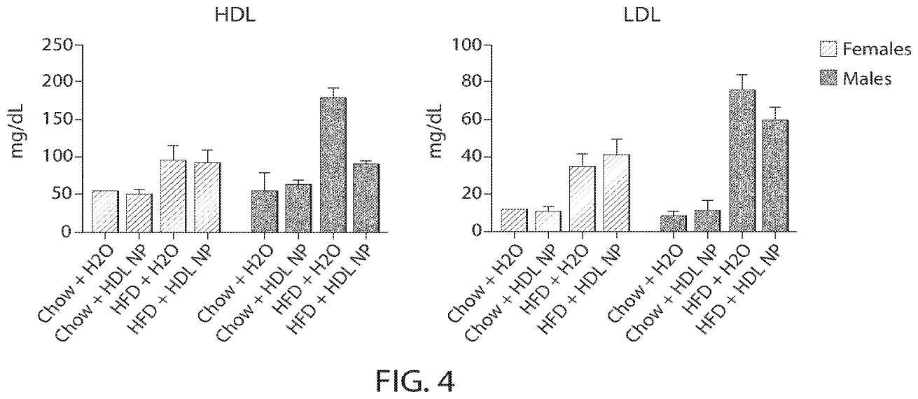

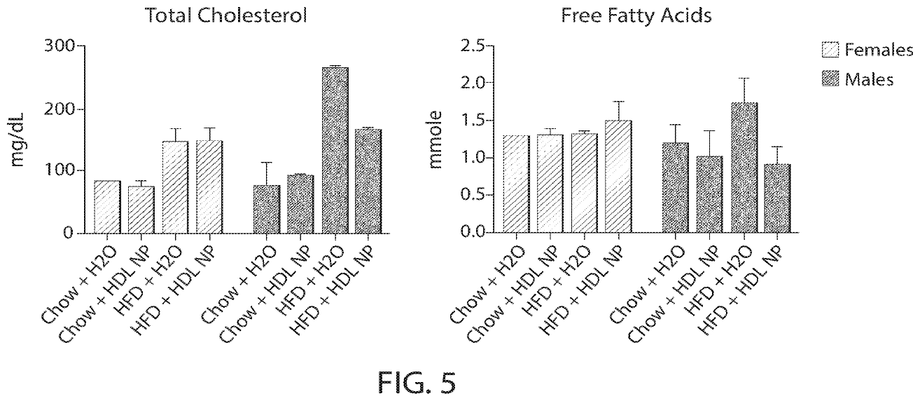

Serum lipid panels were conducted on the male and female mice. The results showed that the HFD increases HDL and low density lipoprotein (LDL) lipid levels in female and male mice. The magnitude of increase was greater in male than female mice. HDL-NP treatment reduced HDL and LDL levels in male mice (FIG. 4). The results also showed that the HFD increased total cholesterol in female and male mice. HDL NP treatment reduced total cholesterol and free fatty acids in HFD-fed male mice (FIG. 5).

### Example 5

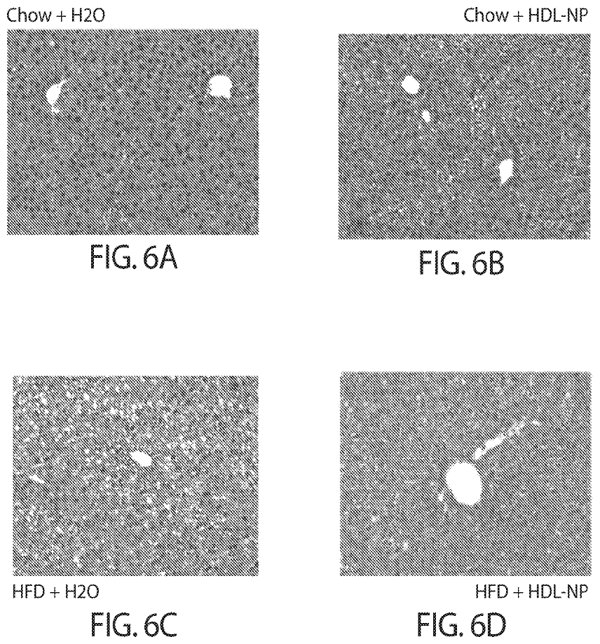

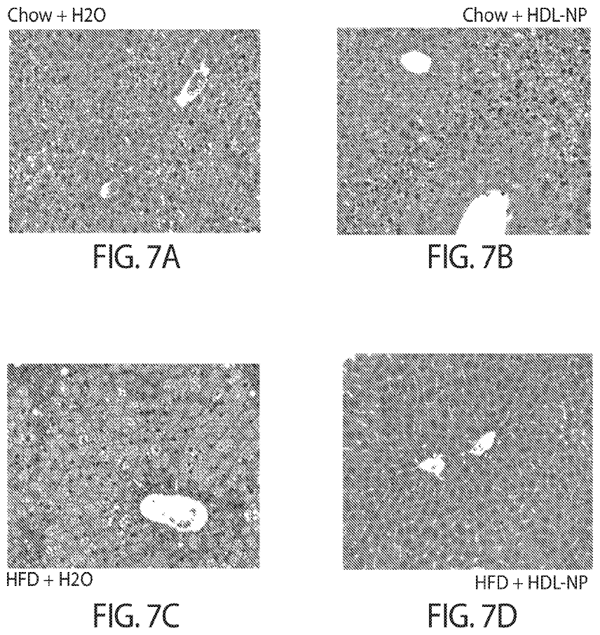

The livers of the male and female mice were sectioned and H&E stained after treatment. The H&E sections were imaged at 20× magnification. The results showed the female mice were protected from HFD-induced steatosis when on an HFD (FIGS. 6A-6D). In contrast, the male mice exhibited HFD-induced steatosis, but the condition was completely resolved in male mice treated with the HDL NP (FIGS. 7A-7D).

### Example 6

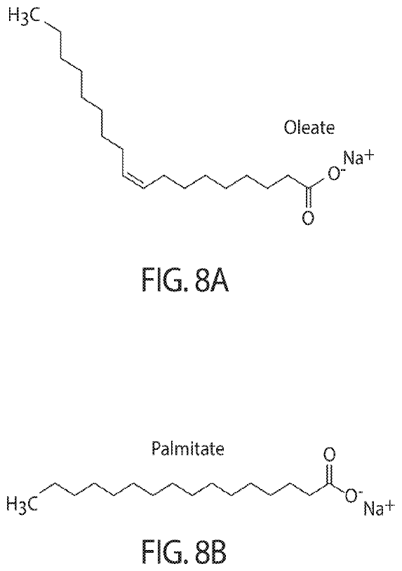

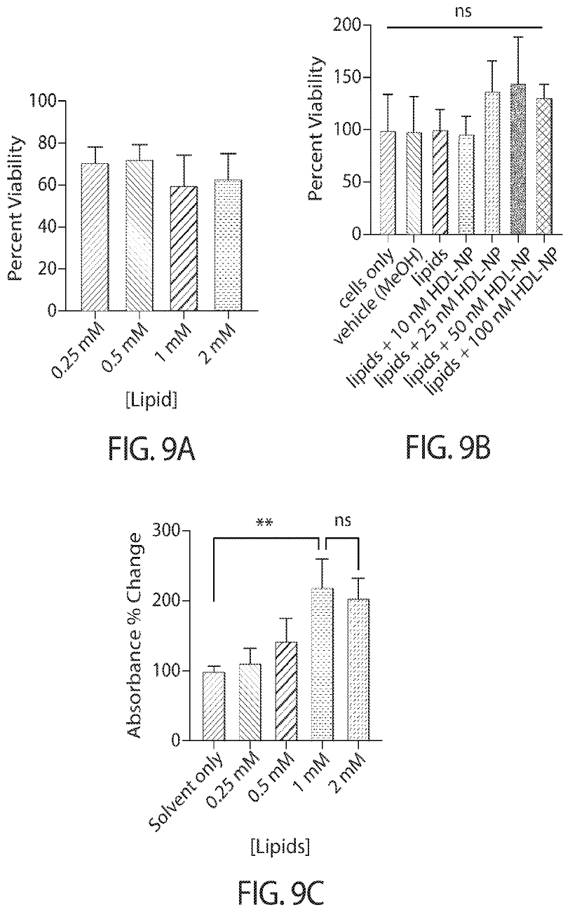

A model of non-alcoholic fatty liver disease (NAFLD) was constructed. Oleic acid (FIG. 8A) was dissolved in 10% bovine serum albumin (BSA) and phosphate buffered-saline (PBS). Palmitate (FIG. 8B) was dissolved in methanol (MeOH). MTS assay w/varying amounts of lipids and thus varying amounts of MeOH to ensure cells remained viable (FIG. 9A). Each lipid was dissolved in MeOH and compared against controls and varying concentrations of lipids with HDL-NPs (FIG. 9B). No significant difference between any of the samples compared to untreated cells by t-test ([HDL-NP]>25 have p-values between 0.1 and 0.05) (FIG. 9B). Lipid accumulation was measured with Oil O Red. The difference between solvent only and 1 mM was significant with p=0.0064. Thus 1 mM total lipids were used for future experiments.

### Example 7

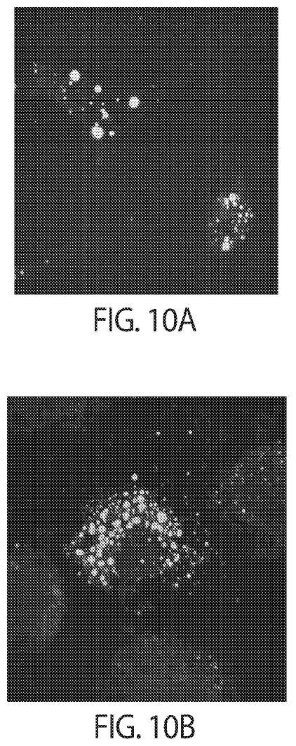

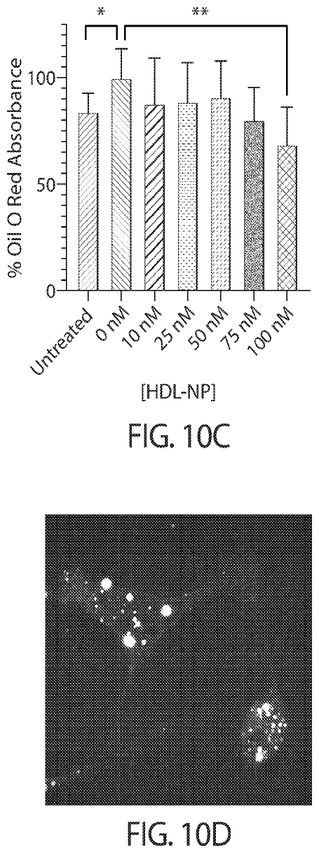

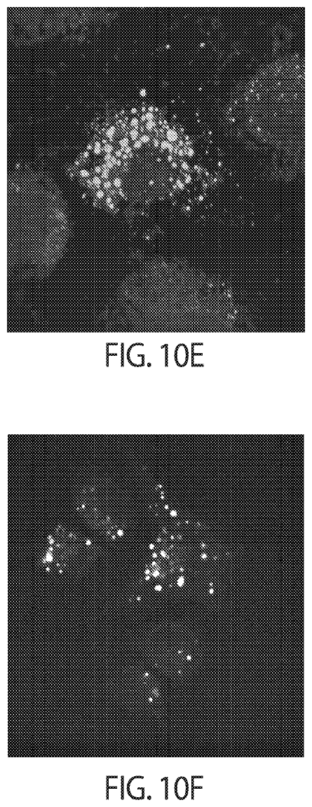

HepG2 cells were stained with Nile Red, without lipids (FIG. 10A) and with lipids (FIG. 10B), incubated for 24 hours, and visualized with fluorescence microscopy. HepG2 cells were incubated with lipids and varying concentrations of HDL-NPs for 24 hours and stained with Oil O Red. Significantly more lipid accumulation (* p=0.0357) was observed in the 0 nM sample (FIG. 10C). Confocal microscopy shows the difference between HepG2 cells incubated for 24 hrs without lipids and without HDL-NPs (FIG. 10D), with lipids and without HDL-NPs (FIG. 10E), and with lipids and with HDL-NPs (FIG. 10F). A marked reduction in intracellular lipid vesicles with HDL-NP treatment (100 nM), which returns cells to a more normal phenotype, is shown.

### OTHER EMBODIMENTS

Embodiment 1. A method for treating a disorder associated with high fat in a subject, comprising orally administering to the subject a synthetic nanostructure comprising: a nanostructure core, an apolipoprotein, a shell comprising a lipid surrounding and attached to the nanostructure core, wherein the shell comprises a phospholipid, wherein the synthetic nanostructure is administered in an effective amount to interact with receptors in the gut endothelium, thereby treating the disorder.

Embodiment 2. The method of embodiment 1, wherein the apolipoprotein is apolipoprotein A-I, apolipoprotein A-II, or apolipoprotein E.

Embodiment 3. The method of any one of the preceding embodiments, further comprising a cholesterol.

Embodiment 4. The method of any one of the preceding embodiments, wherein the shell substantially surrounds the nanostructure core.

Embodiment 5. The method of any one of the preceding embodiments, wherein the shell comprises a lipid monolayer.

Embodiment 6. The method of any one of embodiments 1-4, wherein the shell comprises a lipid bilayer.

Embodiment 7. The method of any one of the preceding embodiments, wherein the shell comprises 50-200 phospholipids.

Embodiment 8. The method of any one of embodiments 1-6, wherein the shell comprises at least 71 phospholipids or 71-95 phospholipids.

Embodiment 9. The method of embodiment 6, wherein at least a portion of the lipid bilayer is covalently bound to the core.

Embodiment 10. The method of any one of the preceding embodiments, comprising a protein associated with at least a portion of the structure.

Embodiment 11. The method of any one of the preceding embodiments, wherein the shell comprises a mixed layer of components.

Embodiment 12. The method of any one of the preceding embodiments, wherein the synthetic nanostructure has a largest cross-sectional dimension of less than or equal to about 5 nm.

Embodiment 13. The method of any one of the preceding embodiments, wherein the nanostructure core is an inorganic nanostructure core.

Embodiment 14. The method of any one of the preceding embodiments, wherein the nanostructure core comprises gold.

Embodiment 15. The method of embodiment 3, wherein the cholesterol is esterified cholesterol.

Embodiment 16. The method of embodiment 3, wherein the cholesterol is free cholesterol.

Embodiment 17. The method of any one of embodiments 1-16, wherein the synthetic nanostructure is administered in an effective amount to regulate systemic lipid homeostasis.

Embodiment 18. The method of any one of embodiments 1-17, wherein the disease is steatosis (fatty liver), NASH, cirrhosis; cardiovascular disease; type II DM; metabolic syndrome; depression; or steroid-based cancer.

Embodiment 19. The method of any one of embodiments 1-18, wherein the subject consumes a diet high in saturated fats.

Embodiment 20. The method of any one of embodiments 1-19, wherein the receptors in the gut endothelium are CD36 receptors.

Embodiment 21. The method of any one of embodiments 1-20, wherein the subject is a male subject.

Embodiment 22. The method of embodiment 18, wherein the disease is steatosis.

Embodiment 23. The method of embodiment 22, wherein the synthetic nanostructure is administered in an effective amount to induce complete resolution of steatosis in a male subject.

Embodiment 24. The method of any one of embodiments 1-23, wherein the synthetic nanostructure is not absorbed or systemically distributed to the liver.

Embodiment 25. A pharmaceutical composition, comprising: a synthetic nanostructure of any one of the preceding embodiments and a pharmaceutically acceptable excipient formulated in an oral dosage form.

Embodiment 26. The pharmaceutical composition of embodiment 25, wherein the oral dosage form is a capsule or tablet.

Embodiment 27. The pharmaceutical composition of embodiment 25, wherein the oral dosage form is a liquid.

Embodiment 28. The method of embodiment 1, wherein the disease is associated with a cancer.

Embodiment 29. The method of embodiment 1, wherein the disease is associated with inflammation.

Embodiment 30. The method of embodiment 1, wherein the disease is associated with prostate cancer.

Embodiment 31. The method of embodiment 1, wherein the disease is associated with cardiovascular disease.

Embodiment 32. The method of embodiment 1, wherein the disease is associated with nonalcoholic steatohepatitis (NASH).

Embodiment 33. The method of any of embodiment 1-24, wherein the disease is not cardiovascular disease, sepsis, pancreatitis, non-alcoholic steatohepatitis, retinopathy, psoriasis, impotence, obesity, diabetes, ichthyosis, stroke, cancer, cataracts, protein storage diseases, disseminated intravascular coagulation, thrombocytopenia, rheumatic diseases, or neurological diseases.

Embodiment 34. A method for reducing fatty acid accumulation in a subject fed a high fat diet, comprising orally administering to the subject a synthetic HDL nanostructure, wherein the synthetic HDL nanostructure is administered in an effective amount to reduce fatty acid accumulation in the subject.

Embodiment 35. A method for treating steatosis in a subject, comprising orally administering to the subject having steatosis, a synthetic HDL nanostructure, wherein the synthetic HDL nanostructure is administered in an effective amount to treat steatosis in the subject.

Embodiment 36. A method for delivering a synthetic HDL nanostructure locally to a gut endothelial tissue of a subject, comprising orally administering to the subject, a synthetic HDL nanostructure, wherein the local delivery of the synthetic HDL nanostructure is restricted to the gut endothelium tissue and wherein the nanostructure is not delivered systemically including to liver tissue in the subject.

Embodiment 37. A method for blocking fatty acid uptake by a scavenger receptor expressed on gut endothelial cells, comprising contacting the scavenger receptor with a synthetic HDL nanostructure in the presence of fatty acids, wherein the synthetic HDL nanostructure binds to the scavenger receptor and blocks fatty acid uptake.

Embodiment 38. The embodiment of any of the methods disclosed herein wherein the synthetic HDL nanostructure comprises a nanostructure core, an apolipoprotein, a shell comprising a lipid surrounding and attached to the nanostructure core, and wherein the shell comprises a phospholipid.

Embodiment 39. The embodiment of any of the methods disclosed herein wherein the subject has a neurological disorder.

Embodiment 40. The embodiment of any of the methods disclosed herein wherein the neurological disorder is depression.

Embodiment 41. The embodiment of any of the methods disclosed herein wherein the subject has cancer.

Embodiment 42. The embodiment of any of the methods disclosed herein wherein the cancer is a hormone driven cancer.

Embodiment 43. The embodiment of any of the methods disclosed herein wherein the hormone driven cancer is prostate cancer.

Embodiment 44. The embodiment of any of the methods disclosed herein wherein the subject has a liver disease.

Embodiment 45. The embodiment of any of the methods disclosed herein wherein the liver disease is cirrhosis.

Embodiment 46. The embodiment of any of the methods disclosed herein wherein the liver disease is steatohepatitis.

Embodiment 47. The embodiment of any of the methods disclosed herein wherein the liver disease is hepatocellular carcinoma.

Embodiment 48. The embodiment of any of the methods disclosed herein wherein the subject has type II diabetes mellitus.

Embodiment 49. The embodiment of any of the methods disclosed herein wherein the subject has metabolic syndrome.

Embodiment 50. The embodiment of any of the methods disclosed herein wherein the subject has pre-eclampsia.

Embodiment 51. The embodiment of any of the methods disclosed herein wherein the subject has polycystic ovarian disease (PCOD).

Embodiment 52. The embodiment of any of the methods disclosed herein, wherein the synthetic HDL nanostructure selectively binds to scavenger receptor expressed on gut endothelial cells.

Embodiment 53. The method of embodiment 52, wherein the scavenger receptor is CD36.

Embodiment 54. The method of embodiment 53, wherein the scavenger receptor is SR-B1.

Embodiment 55. The method of any one of embodiments 35-39, wherein the synthetic HDL nanostructure further comprises a medicament for treating a gastrointestinal tract disorder.

Embodiment 56. The method of any one of embodiments 35-37 and 39, wherein the method further comprises a step of identifying the subject as a subject having a disorder associated with high fat and in need of treatment with the nanostructure.

Embodiment 57. A pharmaceutical composition, comprising: a synthetic nanostructure comprising a nanostructure core, an apolipoprotein, a shell comprising a lipid surrounding and attached to the nanostructure core, wherein the shell comprises a phospholipid and a pharmaceutically acceptable excipient formulated in an oral dosage form.

Embodiment 58. The pharmaceutical composition of embodiment 57, wherein the oral dosage form is a capsule or tablet.

Embodiment 59. The pharmaceutical composition of embodiment 57, wherein the oral dosage form is a liquid.

Embodiment 60. A liquid formulation, comprising a synthetic HDL nanostructure and a liquid carrier suitable for use as an oral dosage form.

Embodiment 61. A solid formulation, comprising a synthetic HDL nanostructure and a liquid carrier suitable for use as an oral dosage form.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method for treating a disorder associated with high fat in a subject, comprising orally administering to the subject a synthetic nanostructure comprising:
a nanostructure core, an apolipoprotein, a shell comprising a lipid surrounding and attached to the nanostructure core, wherein the shell comprises a phospholipid, wherein the synthetic nanostructure is administered in an effective amount to interact with receptors in the gut endothelium, thereby treating the disorder.

2. The method of claim 1, wherein the apolipoprotein is apolipoprotein A-I, apolipoprotein A-II, or apolipoprotein E.

3. The method of any one of the preceding claims, wherein the nanostructure further comprises a cholesterol.

4. The method of any one of the preceding claims, wherein the shell substantially surrounds the nanostructure core.

5. The method of any one of the preceding claims, wherein the shell comprises a lipid monolayer.

6. The method of any one of claims 1-4 wherein the shell comprises a lipid bilayer.

7. The method of any one of the preceding claims, wherein the shell comprises 50-200 phospholipids.

8. The method of any one of claims 1-6, wherein the shell comprises at least 71 phospholipids or 71-95 phospholipids.

9. The method of claim 6, wherein at least a portion of the lipid bilayer is covalently bound to the core.

10. The method of any one of the preceding claims, comprising a protein associated with at least a portion of the structure.

11. The method of any one of the preceding claims, wherein the shell comprises a mixed layer of components.

12. The method of any one of the preceding claims, wherein the synthetic nano structure has a largest cross-sectional dimension of less than or equal to about 5 nm.

13. The method of any one of the preceding claims, wherein the nanostructure core is an inorganic nanostructure core.

14. The method of any one of the preceding claims, wherein the nanostructure core comprises gold.

15. The method of claim 3, wherein the cholesterol is esterified cholesterol.

16. The method of claim 3, wherein the cholesterol is free cholesterol.

17. The method of any one of claims 1-16, wherein the synthetic nanostructure is administered in an effective amount to regulate systemic lipid homeostasis.

18. The method of any one of claims 1-17, wherein the disease is steatosis (fatty liver), NASH, cirrhosis; cardiovascular disease; type II DM; metabolic syndrome; depression; or steroid-based cancer.

19. The method of any one of claims 1-17, wherein the disease is non-alcoholic fatty liver disease (NAFLD).

20. The method of any one of claims 1-17, wherein the disease is not non-alcoholic fatty liver disease (NAFLD).

21. The method of any one of claims 1-17, wherein the disease is not a disease involving reverse cholesterol transport or cardiovascular disease.

22. The method of any one of claims 1-21, wherein the subject consumes a diet high in saturated fats.

23. The method of any one of claims 1-22, wherein the receptors in the gut endothelium are CD36 receptors.

24. The method of any one of claims 1-23, wherein the subject is a male subject.

25. The method of claim 18, wherein the disease is steatosis.

26. The method of claim 22, wherein the synthetic nanostructure is administered in an effective amount to induce complete resolution of steatosis in a male subject.

27. The method of any one of claims 1-23, wherein the synthetic nanostructure is not absorbed or systemically distributed to the liver.

28. The method of claim 1, wherein the disease is associated with a cancer.

29. The method of claim 1, wherein the disease is associated with inflammation.

30. The method of claim 1, wherein the disease is associated with prostate cancer.

31. The method of claim 1, wherein the disease is associated with cardiovascular disease.

32. The method of claim 1, wherein the disease is associated with nonalcoholic steatohepatitis (NASH).

33. The method of any one of claims 1-32, wherein the method further comprises a step of identifying the subject as a subject having a disorder associated with high fat and in need of treatment with the nanostructure.

34. The method of claim 1, wherein the method further comprises a step of identifying the subject as a subject having a fatty liver disease and in need of treatment with the nanostructure.

35. A method for reducing fatty acid accumulation in a subject fed a high fat diet, comprising orally administering to the subject a synthetic HDL nanostructure, wherein the synthetic HDL nanostructure is administered in an effective amount to reduce fatty acid accumulation in the subject.

36. A method for treating steatosis in a subject, comprising orally administering to the subject having steatosis, a synthetic HDL nanostructure, wherein the synthetic HDL nanostructure is administered in an effective amount to treat steatosis in the subject.

37. A method for delivering a synthetic HDL nanostructure locally to a gut endothelial tissue of a subject, comprising orally administering to the subject, a synthetic HDL nanostructure, wherein the local delivery of the synthetic HDL nanostructure is restricted to the gut endothelium tissue and wherein the nanostructure is not delivered systemically including to liver tissue in the subject.

38. A method for blocking fatty acid uptake by a scavenger receptor expressed on gut endothelial cells, comprising contacting the scavenger receptor with a synthetic HDL nanostructure in the presence of fatty acids, wherein the synthetic HDL nanostructure binds to the scavenger receptor and blocks fatty acid uptake.

39. The method of any one of claims 35-38, wherein the synthetic HDL nanostructure comprises a nanostructure core, an apolipoprotein, a shell comprising a lipid surrounding and attached to the nanostructure core, and wherein the shell comprises a phospholipid.

40. The method of any one of claims 35-37 and 39, wherein the synthetic HDL nanostructure is administered to the subject at the same time as a fatty food.

41. The method of any one of claims 35-37 and 39, wherein the synthetic HDL nanostructure is administered to the subject within 12 hours before a fatty food.

42. The method of any one of claims 35-37 and 39, wherein the synthetic HDL nanostructure is administered to the subject within 12 hours after a fatty food.

43. The method of any one of claims 35-37 and 39, wherein the synthetic HDL nanostructure is mixed with a fatty food and administered to the subject with the fatty food.

44. The method of any one of claims 35-37 and 39, wherein the synthetic HDL nanostructure is administered to the subject within 2 hours before a fatty food.

45. The method of any one of claims 35-37 and 39, wherein the synthetic HDL nanostructure is administered to the subject within 2 hours after a fatty food.

46. The method of any one of claims 35-37 and 39, wherein the synthetic HDL nanostructure is administered to the subject once a day.

47. The method of any one of claims 35-37 and 39, wherein the synthetic HDL nanostructure is administered to the subject twice a day.

48. The method of any one of claims 35-37 and 39, wherein the synthetic HDL nanostructure is administered to the subject once every other day.

49. The method of any one of claims 35-37 and 39, wherein the synthetic HDL nanostructure is administered to the subject once a week.

50. The method of any one of claims 35-37 and 39, wherein the synthetic HDL nanostructure is administered to the subject twice a day.

51. The method of any one of claims 35-37 and 39, wherein the synthetic HDL nanostructure is administered to the subject once a day for one week to one month.

52. The method of any one of claims 35-37 and 39, wherein the synthetic HDL nanostructure selectively binds to scavenger receptor expressed on gut endothelial cells.

53. The method of claim 52, wherein the scavenger receptor is CD36.

54. The method of claim 53, wherein the scavenger receptor is SR-B1.

55. The method of any one of claims 35-39, wherein the synthetic HDL nanostructure further comprises a medicament for treating a gastrointestinal tract disorder.

56. The method of any one of claims 35-37 and 39, wherein the method further comprises a step of identifying the subject as a subject having a disorder associated with high fat and in need of treatment with the nanostructure.

57. A pharmaceutical composition, comprising:
a synthetic nanostructure comprising a nanostructure core, an apolipoprotein, a shell comprising a lipid surrounding and attached to the nanostructure core, wherein the shell comprises a phospholipid and a pharmaceutically acceptable excipient formulated in an oral dosage form.

58. The pharmaceutical composition of claim 57, wherein the oral dosage form is a capsule or tablet.

59. The pharmaceutical composition of claim 57, wherein the oral dosage form is a liquid.

60. A liquid formulation, comprising a synthetic HDL nanostructure and a liquid carrier suitable for use as an oral dosage form.

61. A solid formulation, comprising a synthetic HDL nanostructure and a liquid carrier suitable for use as an oral dosage form.
